(19) United States
(12) Patent Application Publication
(10) Pub. No.: US 2003/0138845 A1
(43) Pub. Date: Jul. 24, 2003

(54) Protein and peptide sensors using electrical detection methods

(76) Inventors: Changming Li, Schaumburg, IL (US); Jaymie Sawyer, La Mesa, CA (US); Vi-En Choong, San Jose, CA (US); George Maracas, Phoenix, AZ (US); Peiming Zhang, Gilbert, AZ (US)

Correspondence Address: Robin M. Silva, DORSEY & WHITNEY LLP, Suite 3400, Four Embarcadero Center, San Francisco, CA, 94111-4187, US

(21) Appl. No.: 10/226,639

(22) Filed: Aug. 23, 2002

Related U.S. Application Data
(60) Provisional application No. 60/314,611, filed on Aug. 23, 2001.

(51) Int. Cl. G01N 33/53

(52) U.S. Cl. 435/7.1; 204/461

(57) ABSTRACT

The present invention provides an apparatus and methods for the electrical detection of molecular interactions between a probe molecule and target molecule, but without requiring the use of electrochemical or other reporters to obtain measurable signals. The methods can be used for electrical detection of molecular interactions between probe molecules bound to defined regions of an array and protein or peptide target molecules which are permitted to interact with the probe molecules.peptide.

### FIELD OF THE INVENTION

The present invention is directed to microelectrodes and arrays of microelectrodes for the electrical detection of protein or peptide target molecules based on a molecular interaction between a probe and the target molecules.

### BACKGROUND OF THE INVENTION

A number of commonly-utilized biological applications rely on the ability of analytical technologies to readily detect events related to the interaction between probe and target molecules. However, these detection technologies have traditionally utilized radioactive isotopes or fluorescent compounds to monitor probe-target interactions. For example, Potyrailo et al., 1998, Anal. Chem. 70: 3419-25, describe an apparatus and method for detecting interactions between immobilized fluorescently-labeled aptamers and peptide target molecules. Furthermore, while immunoassays offer some of the most powerful techniques for the molecular detection of peptides, the most sensitive of these techniques requires the use of a fluorescently-, radioactively-labeled, or enzyme linked target or probe molecule.

Methods for the electrical or electrochemical detection of probe-target interactions have provided an attractive alternative to detection techniques relying on radioactive or fluorescent labels. Electrical or electrochemical detection techniques are based on the detection of alterations in the electrical properties of an electrode arising from interactions between one group of molecules attached to the surface of an electrode (often referred to as “probe” molecules) and another set of molecules present in a reaction mixture (often referred to as “target” molecules). Electrical or electrochemical detection eliminates many of the disadvantages inherent in use of radioactive or fluorescent labels to detect interactions between the probe and target molecules. Electrochemical detection offers, for example, a detection technique that is safe, inexpensive, and sensitive, and is not burdened with complex and onerous regulatory requirements.

However, despite these advantages, there are a number of obstacles in using electrical or electrochemical detection techniques for analyzing molecular interactions. One such obstacle is the requirement, in some methods, of incorporating an electrochemical label into the target molecule. Labeled target molecules have been used to increase the electrical signal, thereby permitting molecular interactions between the target molecules and probe molecules to be more readily detected and at lower target concentrations. For example, Meade et al. (in U.S. Pat. Nos. 5,591,578. 5,705,348. 5,770,369, 5,780,234 and 5,824,473) provide methods for the selective covalent modification of target molecules with redox-active moieties such as transition metal complexes. Meade et al. further disclose assays for detecting molecular interactions that employ such covalently-modified target molecules.

Certain alternative methods that do not employ labeled target molecules have been described in the prior art. For example, Hollis et al. (in U.S. Pat. Nos. 5,653,939 and 5,846,708) provide a method and apparatus for identifying molecular structures within a sample substance using a monolithic array of test sites formed on a substrate upon which the sample substance is applied. In the method of Hollis et al., changes in the electromagnetic or acoustic properties—for example, the change in resonant frequency—of the test sites following the addition of the sample substance are detected in order to determine which probes have interacted with target molecules in the sample substance. In addition, Eggers et al. (in U.S. Pat. Nos. 5,532,128, 5,670,322, and 5,891,630) provide a method and apparatus for identifying molecular structures within a sample substance. In the method of Eggers et al. a plurality of test sites to which probes have been bound is exposed to a sample substance, and then an electrical signal is applied to the test sites. Changes in the dielectrical properties of the test sites are subsequently detected to determine which probes have interacted with target molecules in the sample substance.

Another obstacle in the development of a simple and cost-effective electrical and electrochemical detection apparatus for detecting molecular interactions involves limitations in how probe molecules have been attached to electrodes. This is particularly important in fabricating arrays of probes, such as microarrays known in the art. For example, the prior art provides microarrays using polyacrylamide pads for attachment of oligonucleotide probes to a solid support. However, the art has not provided such pads in conjunction with electrodes in an electrical or electrochemical detection apparatus.

Yang et al., 1997, Anal. Chim. Acta 346: 259-75 describe fabrication of microarrays having immobilized probe molecules wherein molecular interactions between labeled target molecules and probes that have been directly attached to solid electrodes are detected using electrical or electrochemical means. Yang et al., however do not suggest using electrical or electrochemical detection techniques in combination with the immobilization of probes on polyacrylamide gel pads. There remains a need in the art to develop alternatives to current detection methods used to detect interactions between biological molecules. More particularly, there is a need in the art to develop electrical and/or electrochemical detection methods for detecting interactions between biological molecules that do not require modifying target or probe molecules with reporter labels. The development of such methods would have wide application in the medical, genetic, and molecular biological arts. There further remains a need in the art to develop alternative methods for attaching biological probe molecules to the microelectrodes of an electrical or electrochemical device.

### SUMMARY OF THE INVENTION

An embodiment of the present invention is directed to methods for the electrical detection of the presence of a target molecule based upon a molecular interaction between a probe molecule the target molecule. An electrical property of a microelectrode is measured, wherein the microelectrode is in contact with a conductive polymer matrix having a molecular probe immobilized thereto. The microelectrode is exposed to a sample mixture suspected of containing the target molecule. The electrical property of said microelectrode is measured again. The two measurements are compared to determining whether a molecular interaction between the probe and the target molecule occurred, which will confirm whether the target molecule is present in the sample mixture. Preferably the electrical property is impedance. Additionally, the probe molecule may be immobilized within the polymer matrix, either covalently or non-covalently, or covalently immobilized on the polymer matrix. The probe molecule may be a protein or peptide. The target molecule may be a protein or a peptide or a small molecule. The probe molecule may also be a small molecule, although this is not preferred. In a preferred embodiment the present invention does not use a reporter, electrochemical or otherwise, attached to probe or target molecule.

### DESCRIPTION OF THE DRAWINGS

FIGS. 1A and 1B illustrate a schematic representation of the structure of a microelectrode in contact with a polyacrylamide gel, or polypyrrole linker moiety (i.e., a porous hydrogel microelectrode) (FIG. 1A) and a schematic representation of the structure of the tip of the porous hydrogel microelectrode (FIG. 1B);

FIG. 2 illustrates a porous hydrogel microelectrode;

FIGS. 3A and 3B illustrate plots of capacitance versus frequency (FIG. 3A) and resistance versus frequency (FIG. 3B) for a streptavidin-modified porous hydrogel microelectrode before (curve 1) and after immobilization of rabbit anti-BAP antibody (curve 2), and following incubation with BAP antigen (curve 3); and

FIGS. 4A and 4B illustrate plots of capacitance versus frequency (FIG. 4A) and resistance versus frequency (FIG. 4B) for a streptavidin-modified porous hydrogel microelectrode with immobilized rabbit anti-BAP antibody before (curve 1) and after incubation with BAP antigen (curve 2), and following incubation with an anti-rabbit IgG secondary antibody (curve 3).

FIG. 5 illustrates a plot of the AC impedance responses of an electrode in contact with a polypyrrole film embedded with unconjugated rabbit anti-BAP in PBS buffer at 0.1 M Na⁺ (curve 1), 0.5 M Na⁺ (curve 2), 1.0M Na⁺ (curve 3), and 2.0 M Na⁺ (curve 4);

FIG. 6 plots Faraday resistance versus the concentration of Na⁺ derived from the curves of FIG. 5;

FIG. 7 plots the AC impedance responses of an electrode in contact with a polypyrrole film embedded with unconjugated rabbit anti-BAP (Bacterial Alkaline Phosphatase), which electrode was incubated in PBS buffer (curve 1), and incubated in PBS buffer with BAP (curve 2);

FIG. 8 plots the AC impedance responses of an electrode in contact with a polypyrrole film which electrode was incubated in PBS buffer (curve 1), and incubated in PBS buffer with BAP (curve 2), and the AC impedance responses of an electrode in contact with a polypyrrole film embedded with unconjugated rabbit anti-BAP, which electrode was incubated in PBS buffer (curve 3, curve 1 of FIG. 7), and incubated in PBS buffer with BAP (curve 4, curve 2 of FIG. 7);

FIG. 9 plots the AC impedance responses of an electrode in contact with a polypyrrole film embedded with unconjugated anti-GP 120, which electrode was incubated in PBS buffer (curve 1), and incubated in PBS buffer with GP120, an outer envelope protein of HIV-I (curve 2);

FIG. 10 plots the AC impedance responses of an electrode in contact with a polypyrrole film embedded with anti-AZT, which electrode was incubated in PBS buffer (curve 1), and incubated in PBS buffer with AZT (curve 2);

FIG. 11 plots the AC impedance responses of an electrode in contact with a polypyrrole film embedded with anti-GP105, which electrode was incubated in PBS buffer (curve 1), and incubated in PBS buffer with GP105, an outer envelope protein of HIV-II (curve 2);

FIG. 12 plots the average of the difference in Faraday resistance of a baseline and that observed after incubation of the electrode in a target solution versus concentration of the target; and

FIG. 13 plots the average of the difference in resistance of a baseline and that observed after incubation of the electrode in one of two different target solutions versus the inverse of the square root of the frequency.

### DETAILED DESCRIPTION OF THE PREFERRED EMBODIMENTS

The apparatus and methods of the present invention are useful for the electrical detection of molecular interactions between probe molecules that are immobilized by linker moieties or immobilized within the matrix of the linker moieties in contact with microelectrodes and protein or peptide target molecules in a sample mixture. In a preferred embodiment neither the probe nor target molecule have a reporter attached thereto.

As used herein, the term “array” refers to an ordered spatial arrangement, particularly an arrangement of immobilized biomolecules, such as specific binding molecules or probes as further described below. The present system finds particular utility in array formats, i.e. wherein there is a matrix of addressable locations (herein generally referred to as “pads”, “addresses” or “micro-locations”). “Array” or grammatical equivalents thereof as used herein mean a plurality of probes in an array format; the size of the array will depend on the composition and end use of the array. Arrays containing from about 2 different probes to many thousands can be made. Generally, the array will comprise from two to as many as 100,000 or more per cm², depending on the size of the pads, as well as the end use of the array. Preferred ranges are from about 2 to about 10,000, with from about 5 to about 1000 being preferred, and from about 10 to about 100 being particularly preferred. In some embodiments, the compositions of the invention may not be in array format; that is, for some embodiments, compositions comprising a single probe may be made as well. In addition, in some arrays, multiple substrates may be used, either of different or identical compositions. Thus for example, large arrays may comprise a plurality of smaller substrates.

As used herein, the terms “bioarray,” “biochip” and “biochip array” refer to an ordered spatial arrangement of immobilized biomolecules on a microelectrode arrayed on a solid supporting substrate. Preferred probe molecules include nucleic acids, oligonucleotides, peptides, ligands, antibodies and antigens; peptides and proteins are the most preferred probe species. Biochips, as used in the art, encompass substrates containing arrays or microarrays, preferably ordered arrays and most preferably ordered, addressable arrays, of biological molecules that comprise one member of a biological binding pair. Typically, such arrays are oligonucleotide arrays comprising a nucleotide sequence that is complementary to at least one sequence that may be or is expected to be present in a biological sample. Alternatively, and preferably, proteins, peptides or other small molecules can be arrayed on such biochips for performing, inter alia, immunological analyses (wherein the arrayed molecules are antigens), assaying biological receptors (wherein the arrayed molecules are ligands, agonists or antagonists of said receptors). In alternative embodiments, the probes of the invention are specific for a particular species, subspecies or strain of bacteria, and most preferably are specific for viable bacteria of said species, subspecies or strain.

As used herein, the term “addressable array” refers to an array wherein the individual elements have precisely defined x and y coordinates, so that a given element at a particular position in the array can be identified.

As used herein, the terms “probe”, “biomolecular probe”, “binding molecules”, “binding ligand”, or grammatical equivalents thereof refer to a molecule (preferably a biomolecule) used to detect another biomolecule. Examples include antigens that detect antibodies, oligonucleotides that detect complimentary oligonucleotides, and ligands that detect receptors. Such probes are preferably immobilized on a microelectrode comprising a substrate.

“Binding ligand” or grammatical equivalents thereof as used herein means a compound that is used to probe for the presence of the target analyte, and that will bind to the target analyte. As will be appreciated by those in the art, the composition of the binding ligand will depend on the composition of the target analyte. Binding ligands for a wide variety of analytes are known or can be readily found using known techniques. For example, when the analyte is a protein, the binding ligands include proteins (particularly including antibodies or fragments thereof (FAbs, etc.)) or small molecules. When the analyte is a metal ion, the binding ligand generally comprises traditional metal ion ligands or chelators. Preferred binding ligand proteins include peptides. For example, when the analyte is an enzyme, suitable binding ligands include substrates and inhibitors or visa versa. Antigen-antibody pairs, receptor-ligands, and carbohydrates and their binding partners are also suitable analyte-binding ligand pairs. The binding ligand may be nucleic acid, when nucleic acid binding proteins are the targets; alternatively, as is generally described in U.S. Pat. Nos. 5,270,163, 5,475,096, 5,567,588, 5,595,877, 5,637,459, 5,683,867,5,705,337, and related patents (hereby incorporated by reference) nucleic acid “aptomers” can be developed for binding to virtually any target analyte. Similarly, there is a wide body of literature relating to the development of binding partners based on combinatorial chemistry methods. In this embodiment, when the binding ligand is a nucleic acid, preferred compositions and techniques are outlined in PCT US97/20014, hereby incorporated by reference. As used herein “nucleic acid”, “oligonucleotide” or grammatical equivalents mean at least two nucleotides covalently linked together. A nucleic acid according to the present invention will generally contain phosphodiester bonds, although in some cases nucleic acid analogs are included that may have alternate backbones, comprising, for example, phosphoramide (Beaucage et al., Tetrahedron 49(10):1925 (1993); Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemical Scripta 26:141 (1986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989)), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996)) (all of these references are incorporated herein in their entirety by reference). Other analog nucleic acids include those with positive backbones (Denpcy et al., Proc. Natl. Acad. Sci. USA 92:6097 (1995)), non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)), and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. As will be appreciated by those in the art, all of these nucleic acid analogs may find use in the present invention.

In a preferred embodiment, the binding of the target analyte to the binding ligand is specific, and the binding ligand is part of a binding pair. As used herein “specifically bind” means that the ligand binds the analyte with specificity sufficient to differentiate between the analyte and other components or contaminants within the test sample. However, as will be appreciated by those in the art, it will be possible to detect analytes using binding which is not highly specific; for example, the systems may use different binding ligands, for example an array of different ligands, and detection of any particular analyte is via its “signature” of binding to a panel of binding ligands, similar to the manner in which “electronic noses” work. This finds particular utility in the detection of chemical analytes. The binding should be sufficient to remain bound under the conditions of the assay, including wash steps to remove non-specific binding. In some embodiments, for example in the detection of certain biomolecules, the disassociation constants of the analyte to the binding ligand will be less than about 10⁻⁴-10⁻⁶ M⁻¹, with less than about 10⁻⁵ to 10⁻⁹ M⁻¹ being preferred and less than about 10⁻⁷-10⁻⁹ M⁻¹ being particularly preferred.

In a preferred embodiment probes or targets may be proteins. As used herein “peptides”, “polypeptides”, “proteins” or grammatical equivalents thereof mean proteins, oligopeptides, peptides, derivatives and analogs thereof (including without limitation proteins containing non-naturally occurring amino acids and amino acid analogs), and peptidomimetic structures. The side chains may be in either the (R) or the (S) configuration. In a preferred embodiment, the amino acids are in the (S) or L-configuration. When the protein is used as a binding ligand, it may be desirable to utilize protein analogs to retard degradation by sample contaminants.

In some embodiments of the present invention, the probe molecules of the apparatus comprise proteins or peptides. The protein or peptide probe molecules of the present invention are preferably peptides comprising from about 5 to about 100 amino acids, more preferably antigen-recognizing peptieds or polypeptides belonging to the immunoglobulin superfamily. Said peptide or polypeptide probe molecules are immobilized to the microelectrodes of the invention through linker moieties, using techniques known to those with skill in the art wherein said linkage does not interfere with or inhibit the ability of the probe molecules to interact with protein or peptide target molecules in the sample mixture. As used herein “immobilize” or grammatical equivalents thereof means that the probe molecule is fixed relative to the microelectrode. For example and without limitation a probe molecule may be attached to a linker moiety, the probe molecule may be embedded within a matrix of a linker moiety, or any combination thereof, and the linker moiety is attached to the microelectrode. It will be appreciated that direct attachment of the probe molecule to the electrode is possible; however, this is not preferred. Attachment of the probe to a linker moiety is well within the skill of the artisan, and may include without limitation covalent bonding, ionic bonding, van der Waals forces, hydro-phobic/philic interactions, biologic attachment (such as avidin-streptavidin interactions), the latter being preferred. Additionally, the probe molecule may be embedded within the matrix of the linker moiety. For example the probe molecule may be mixed with a monomer followed by polymerization, which immobilizes the probe molecule within the polymer matrix. Alternatively, the probe molecule may be covalently attached to the monomer, and polymerization of the monomer immobilizes the probe molecule within the polymer matrix. In one preferred embodiment, discussed more thoroughly below and in the Examples the polymer is a conductive polymer.

In a preferred embodiment, the method of attachment of the proteins serving as capture binding ligands is through the use of an attachment linker. This will generally be done as is known in the art, and will depend on both the composition of the attachment linker and the capture binding ligand. In general, the capture binding ligands are attached to the attachment linker through the use of functional groups on each that can then be used for attachment. Similarly, if an attachment linker is used, functional groups on the conductive polymer can be used as well. Preferred functional groups for attachment are amino groups, carboxy groups, oxo groups and thiol groups. These functional groups can then be attached, either directly or indirectly through the use of a linker. Linkers are well known in the art; for example, homo-or hetero-bifunctional linkers as are well known (see 1994 Pierce Chemical Company catalog, technical section on cross_linkers, pages 155_{—}200, incorporated herein by reference). Preferred linkers include, but are not limited to, alkyl groups (including substituted alkyl groups and alkyl groups containing heteroatom moieties), with short alkyl groups, esters, amide, amine, epoxy groups and ethylene glycol and derivatives being preferred, with propyl, acetylene, and C2 alkene being especially preferred. The linker may also be a sulfone group, forming sulfonamide linkages. In this way, capture binding ligands comprising proteins can be added, either directly to the polymers or indirectly through an attachment linker, to the electrodes of the array.

In one preferred embodiment antibodies are used as probes. A protein may be used to generate polyclonal and monoclonal antibodies to proteins, which are useful as described herein. In a preferred embodiment, the epitope of the probe is unique; that is, antibodies generated to a unique epitope show little or no cross-reactivity to other proteins. As used herein “epitope”, “determinant”, or grammatical equivalents thereof means a portion of a protein which will generate and/or bind an antibody. Thus, in most instances, antibodies made to a smaller protein will be able to bind to the full length protein. The term “antibody” includes antibody fragments, as are known in the art, including Fab, Fab₂, single chain antibodies (Fv for example), chimeric antibodies, etc., either produced by the modification of whole antibodies or those synthesized de novo using recombinant DNA technologies. The term “antibody” further comprises polyclonal antibodies and monoclonal antibodies, which can be agonist or antagonist antibodies.

The proteins, whether a target or a probe, may be from any organism, including prokaryotes and eukaryotes, with enzymes from bacteria, fungi, extremeophiles such as the archebacteria, insects, fish, animals (particularly mammals and particularly human) and birds all possible. Specifically included within the definition of “protein” are fragments and domains of known proteins, including functional domains such as enzymatic domains, binding domains, etc., and smaller fragments, such as turns, loops, etc. That is, portions of proteins may be used as well. In addition, “protein” as used herein includes proteins, oligopeptides and peptides. In addition, protein variants, i.e. non-naturally occurring protein analog structures, may be used.

Suitable proteins for probes or targets include, but are not limited to, industrial and pharmaceutical proteins, including ligands, cell surface receptors, antigens, antibodies, cytokines, hormones, transcription factors, signaling modules, cytoskeletal proteins and enzymes. Suitable classes of enzymes include, but are not limited to, hydrolases such as proteases, carbohydrases, lipases; isomerases such as racemases, epimerases, tautomerases, or mutases; transferases, kinases, oxidoreductases, and phophatases. Suitable enzymes are listed in the Swiss_Prot enzyme database. Suitable protein backbones include, but are not limited to, all of those found in the protein data base compiled and serviced by the Research Collaboratory for Structural Bioinformatics (RCSB, formerly the Brookhaven National Lab).

Specifically, preferred target proteins include, but are not limited to, those with known structures (including variants) including cytokines (IL-1ra (+receptor complex), IL-1 (receptor alone), IL-1a, IL-1b (including variants and or receptor complex), IL-2, IL-3, IL-4, IL-5, IL-6, IL-8, IL-10, IFN-β, INF-γ, IFN-α-2a; IFN-α-2B, TNF-α; CD40 ligand (chk), Human Obesity Protein Leptin, Granulocyte Colony-Stimulating Factor, Bone Morphogenetic Protein-7, Ciliary Neurotrophic Factor, Granulocyte-Macrophage Colony-Stimulating Factor, Monocyte Chemoattractant Protein 1, Macrophage Migration Inhibitory Factor, Human Glycosylation-Inhibiting Factor, Human Rantes, Human Macrophage Inflammatory Protein 1 Beta, human growth hormone, Leukemia Inhibitory Factor, Human Melanoma Growth Stimulatory Activity, neutrophil activating peptide-2, Cc-Chemokine Mcp-3, Platelet Factor M2, Neutrophil Activating Peptide 2, Eotaxin, Stromal Cell-Derived Factor-1, Insulin, Insulin-like Growth Factor I, Insulin-like Growth Factor II, Transforming Growth Factor B1, Transforming Growth Factor B2, Transforming Growth Factor B3, Transforming Growth Factor α, Vascular Endothelial growth factor (VEGF), acidic Fibroblast growth factor, basic Fibroblast growth factor, Endothelial growth factor, Nerve growth factor, Brain Derived Neurotrophic Factor, Ciliary Neurotrophic Factor, Platelet Derived Growth Factor, Human Hepatocyte Growth Factor, Glial Cell-Derived Neurotrophic Factor, (as well as the 55 cytokines in PDB 1/12/99)); Erythropoietin; other extracellular signaling moieties, including, but not limited to, hedgehog Sonic, hedgehog Desert, hedgehog Indian, hCG; coaguation factors including, but not limited to, TPA and Factor VIIa; transcription factors, including but not limited to, p53, p53 tetramerization domain, Zn fingers (of which more than 12 have structures), homeodomains (of which 8 have structures), leucine zippers (of which 4 have structures); antibodies, including, but not limited to, cFv; viral proteins, including, but not limited to, hemagglutinin trimerization domain and hiv Gp41 ectodomain (fusion domain); intracellular signalling modules, including, but not limited to, SH2 domains (of which 8 structures are known), SH3 domains (of which 11 have structures), and Pleckstin Homology Domains; receptors, including, but not limited to, the extracellular Region Of Human Tissue Factor Cytokine-Binding Region Of Gp130, G-CSF receptor, erythropoietin receptor, Fibroblast Growth Factor receptor, TNF receptor, IL-1 receptor, IL-1 receptor/IL1ra complex, IL-4 receptor, INF-α receptor alpha chain, MHC Class I, MHC Class II, T Cell Receptor, Insulin receptor, insulin receptor tyrosine kinase and human growth hormone receptor. It will be understood fragments and domains of these proteins, including functional domains, may also be used as probes.

In an alternative embodiment, the target molecules are proteins as defined above. In a preferred embodiment, the target molecules are naturally occurring proteins or fragments of naturally occurring proteins. Thus, for example, cellular extracts containing proteins, or random or directed digests of proteinaceous cellular extracts, may be used. In this way libraries of prokaryotic and eukaryotic proteins may be made for screening in accordance with the present invention. Particularly preferred in this embodiment are bacterial, fungal, viral, and mammalian proteins (or libraries thereof), with the latter being preferred, and human proteins being especially preferred.

As will be appreciated, the probe or target molecules may comprise a natural products library, a peptide library, a phage display library, or a combinatorial library.

Suitable target protein molecules include, but are not limited to, (1) immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (such as phenytoin, primidone, carbariezepin, ethosuximide, valproic acid, and phenobarbitol), cardioactive drugs (such as digoxin, lidocaine, procainamide, and disopyramide), bronchodilators (such as theophylline), antibiotics (such as chloramphenicol, sulfonamides), antidepressants, immunosuppresants, abused drugs (such as amphetamine, methamphetamine, cannabinoids, cocaine and opiates), and antibodies to any number of viruses (such as orthomyxoviruses, (e.g., influenza virus), paramyxoviruses (e.g., respiratory syncytial virus, mumps virus, measles virus), adenoviruses, rhinoviruses, coronaviruses, reoviruses, togaviruses (e.g., rubella virus), parvoviruses, poxviruses (e.g., variola virus, vaccinia virus), enteroviruses (e.g., poliovirus, coxsackievirus), hepatitis viruses (including A, B and C), herpesviruses (e.g., Herpes simplex virus, varicella_zoster virus, cytomegalovirus, Epstein_Barr virus), rotaviruses, Norwalk viruses, hantavirus, arenavirus, rhabdovirus (e.g., rabies virus), retroviruses (including HIV, HTLV_I and _II), papovaviruses (e.g., papillomavirus), polyomaviruses, and picornaviruses.), and bacteria (including a wide variety of pathogenic and non_pathogenic prokaryotes of interest including Bacillus; Vibrio, e.g., V. cholerae; Escherichia, e.g., Enterotoxigenic E. coli, Shigella, e.g., S. dysenteriae; Salmonella, e.g., S. typhi; Mycobacterium e.g., M. tuberculosis, M. leprae; Clostridium, e.g., C. botulinum, C. tetani, C. difficile, C.perfringens; Cornyebacterium, e.g., C. diphtheriae; Streptococcus, S. pyogenes, S. pneumoniae; Staphylococcus, e.g., S. aureus; Haemophilus, e.g., H. influenzae; Neisseria, e.g., N. meningitidis, N. gonorrhoeae; Yersinia, e.g., G. lambliaY. pestis, Pseudomonas, e.g., P. aeruginosa, P. putida; Chlamydia, e.g., C. trachomatis; Bordetella, e.g., B. pertussis; Treponema, e.g., T. palladium; and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphotase; aldolase, prostatic acid phosphatase, terminal deoxynucleotidyl transferase, and bacterial and viral enzymes such as HIV protease; (3) hormones and cytokines (many of which serve as ligands for cellular receptors) such as erythropoietin (EPO), thrombopoietin (TPO), the interleukins (including IL1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone, testosterone; and (4) other proteins (including α-fetoprotein, carcinoembryonic antigen CEA.

In addition, any of the biomolecules for which antibodies are tested may be tested directly as well; that is, the virus or bacterial cells, therapeutic and abused drugs, etc., may be the test molecules. In preferred embodiments, the apparatus of the present invention comprises a supporting substrate, one or a plurality of microelectrodes in contact with the supporting substrate, one or a plurality of linking moieties in contact with the microelectrodes and to which specific binding molecules are immobilized, at least one counter-electrode in electrochemical contact with the microelectrodes, a means for producing an electrical signal at each microelectrode, a means for detecting changes in the electrical signal at each microelectrode, and an electrolyte solution in contact with the microelectrodes, linking moieties, and counter-electrode.

In some embodiments of the present invention, the specific binding molecules of the apparatus comprise proteins or peptides. In one preferred embodiment, the specific binding molecules are antibodies. The antibodies immobilized on the linker moieties of the apparatus of the invention may be polyclonal or monoclonal antibodies, F(ab) fragments, F(ab)′ fragments, F(ab)₂ fragments, or Fᵥ fragments of polyclonal or monoclonal antibodies, or F(ab) or single chain antibodies selected from in vitro libraries. In alternative embodiments of the present invention, the specific binding molecules are nucleic acids, oligonucleotides, or combinations thereof. In one preferred embodiment of the present invention, the specific binding molecules are aptamers (i.e., oligonucleotides capable of interacting with target molecules such as peptides). Natural products libraries (such as, inter alia, yeast extracts), phage display libraries, or combinatorial libraries may also be used as specific binding molecules.

In one preferred embodiment of the present invention, the specific binding molecules are polyclonal antibodies and the antibodies are immobilized on the linker moieties using any technique known in the art that does not interfere with or inhibit the ability of the antibodies to specifically bind to a target analyte, such as and without limitation the conjugate antigen or portion thereof. In preferred embodiments, polyclonal antibodies, antisera, monoclonal antibodies, fragments of said polyclonal or monoclonal antibodies, or other specific ligand binding molecules, are immobilized onto and attached to the linker moieties of the apparatus of the invention using biotinylated species thereof that are conjugated with streptavidin added to the linker moiety material. In more preferred embodiments, the linker moiety material to which the streptavidin is added is polyacrylamide gel. In other embodiments the biotinylated specific binding molecule is a protein, peptide, nucleic acid, or oligonucleotide. Alternatively and as further described below, the specific binding molecules are embedded within a matrix, e.g., polymer matrix, of the linker molecules.

In some embodiments of the present invention, the linker moieties of the apparatus are composed of materials including, but not limited to, polyacrylamide gel, agarose gel, polyethylene glycol, cellulose gel, or sol gels. In preferred embodiments, the linker moieties comprise polyacrylamide gel, which form a gel pad. In alternative embodiments of the present invention, the linker moieties, comprise a conjugated polymer or copolymer film. Such conjugated polymer or copolymer film is composed of materials including, but not limited to, polypyrrole, polythiphene, polyaniline, polyfuran, polypyridine, polycarbazole, polyphenylene, poly(phenylenvinylene), polyfluorene, or polyindole, or their derivatives, their copolymers, and combinations thereof. In preferred embodiments, the linker moieties comprise a neutral pyrrole matrix, and the probes are embedded therein. In an alternative embodiment the linker moiety is a conductive polymer where the probe may be non-covalently embedded within the polymer matrix, covalently embedded within the polymer matrix or covalently attached to the surface of the polymer matrix. For example, and not by way of limitation, conductive polymers include polypyrrole, polythiphene, polyaniline, polyfuran, polypyridine, polycarbazole, polyphenylene, poly(phenylenevinylene), polyfluorene, polyindole, derivatives thereof, co-polymers thereof, and combinations thereof. Preferably the conductive polymer is polypyrrole, polythiophene and polyaniline, and most preferable is polypyrrole.

Polymeric hydrogels and gel pads are used as binding layers to adhere biological molecules to substrate surfaces, which biomolecules include, but are not limited to, proteins, peptides, oligonucleotides, polynucleotides, and larger nucleic acid fragments. The oligonucleotide probes may be bound to the surface of a continuous layer of the hydrogel, to an array of gel pads, or embedded therein. The gel pads comprising biochips for use with the apparatus of the present invention are conveniently produced as thin sheets or slabs, typically by depositing a solution of acrylamide monomer, a crosslinker such methylene bisacrylamide, and a catalyst such as N, N, N′, N′-tetramethylethylendiamine (TEMED) and an initiator such as ammonium persulfate for chemical polymerization, or 2,2-dimethoxy-2-phenyl-acetophone (DMPAP) for photopolymerization, in between two glass surfaces (e.g., glass plates or microscope slides) using a spacer to obtain the desired thickness of the polymeric gel. Generally, the acrylamide monomer and crosslinker are prepared in one solution of about 4-5% acrylamide (having an acrylamide/bisacrylamide ratio of 19/1) in water/glycerol, with a nominal amount of initiator added. The solution is polymerized and crosslinked either by ultraviolet (UV) radiation (e.g., 254 nm for at least about 15 minutes, or other appropriate UV conditions, collectively termed “photopolymerization”), or by thermal initiation at elevated temperature (e.g., typically at about 40° C.). Following polymerization and crosslinking, the top glass slide is removed from the surface to uncover the gel. The pore size (and hence the “sieving properties”) of the gel is controlled by varying the amount of crosslinker and the percent solids in the monomer solution. The pore size also can be controlled by varying the polymerization temperature.

In the fabrication of arrays of polyacrylamide (i.e., patterned gels), in an embodiment of the present invention, the acrylamide solution typically is imaged through a mask during the UV polymerization/crosslinking step. The top glass slide is removed after polymerization, and the unpolymerized monomer is washed away (developed) with water, leaving a fine feature pattern of polyacrylamide hydrogel, which is used to produce the crosslinked polyacrylamide hydrogel pads. Further, in an application of lithographic techniques known in the semiconductor industry, light can be applied to discrete locations on the surface of a polyacrylamide hydrogel to activate these specified regions for the attachment of an oligonucleotide, an antibody, an antigen, a hormone, hormone receptor, a ligand or a polysaccharide on the surface (e.g., a polyacrylamide hydrogel surface) of a solid substrate (see, e.g, WO 91/07087, incorporated herein by reference).

For hydrogel-based arrays using polyacrylamide, biomolecules (such as oligonucleotides, peptides, polypeptides, or proteins) are covalently attached by forming an amide, ester or disulfide bond between the biomolecule and a derivatized polymer comprising the cognate chemical group. Covalent attachment of the biomolecule to the polymer is usually performed after polymerization and chemical cross-linking of the polymer is completed.

Alternatively, oligonucleotides bearing 5′-terminal acrylamide modifications can be used that efficiently copolymerize with acrylamide monomers to form DNA-containing polyacrylamide copolymers (Rehman et al., 1999, Nucleic Acids Research 27: 649-655). Using this approach, stable probe-containing layers can be fabricated on substrates (e.g., microtiter plates and silanized glass) having exposed acrylic groups. This approach has made available the commercially marketed “Acrydite™” capture probes (available from Mosaic Technologies, Boston, Mass.). The Acrydite moiety is a phosporamidite that contains an ethylene group capable of free-radical copolymerization with acrylamide, and which can be used in standard DNA synthesizers to introduce copolymerizable groups at the 5′ terminus of any oligonucleotide probe.

Alternatively, the linker moieties may be formed using electrochemical techniques. For example, cyclic voltammetry of pyrrole, 3-acetateN-hydroxysuaccinimido pyrrole and PBS buffer, in the presence of probe molecules, forms a polypyrrole/probe film on a microelectrode surface. The probe molecules are embedded or fixed within the film. Other methods of embedding probe molecules within a gel pad matrix will be known to the skilled artisan. For example, polymerization of polyacrylamide gel in the presence of the probe molecules will embed the probe molecules therein. In a preferred embodiment, the linker moiety is a conductive polymer matrix that has the probe molecules non-covalently immobilized within the polymer matrix, covalently immobilized within the polymer matrix, or covalently attached to the surface of the polymer matrix. Conductive polymers include polymers that undergo single or multi-electron oxidation or reduction reaction in an electrochemical cell. The conductive polymers can be prepared in a film on an electrode surface by electrochemical polymerization of the corresponding monomers, using conventional electrochemical methods, such as and without limitation cyclic voltammetry, constant potential deposition. Conductive polymers include without limitation polypyrrole, polythiphene, polyaniline, polyfuran, polypyridine, polycarbazole, polyphenylene, poly(phenylenevinylene), polyfluorene, polyindole, derivatives thereof, co-polymers thereof, and combinations thereof. Preferably the conductive polymer is polypyrrole, polythiophene and polyaniline, and most preferable is polypyrrole.

Immobilization of a probe molecule in the conductive polymer matrix can be accomplished, without limitation, by electrochemically polymerizing the monomer in the presence of the probe molecule. Alternatively, the probe molecule could be covalently attached to monomers by various linkers and the corresponding functional groups known to the skilled artisan, which would then be electrochemically polymerized. The skilled artisan will recognize that the amount of probe molecule can be readily adjusted by varying the ratio of probe to monomer in the polymerization solution. In another alternative, the probe molecules may be covalently attached the surface of the polymerized conductive polymer by various linkers and the corresponding functional groups known to the skilled artisan, and described in Gordon Bickerstaff, Immobilization of Enzymes and Cells (Humana Press 1997), which is incorporated herein by reference in its entirety for all purposes. For example and without limitation the linkers to attach probe molecules to the surface of the conductive polymer or to the monomers of the conductive polymer include, without limitation, NHS-ester, maleimide, imidoester, active halogen, carboxylic acid-EDC, pyridyl disulfide, azidophenyl, vinyl-sulfone, hydrazide, isocyanate.

Low, medium and high density arrays of conductive polymer matrix arrays can be constructed in accordance with an embodiment of the present invention. For example an array of electrodes is manufactured into a substrate, with each electrode being individually addressable, as is known by the skilled artisan. The substrate with electrodes is exposed to a solution of monomer and a first probe molecule, and a first electrode is activated to electrochemically polymerize the conductive polymer onto the electrode surface immobilizing the first probe molecule within the matrix. The substrate is then thoroughly rinsed and exposed to a solution of monomer and a second probe molecule, and a second electrode is activated to electrochemically polymerize the conductive polymer onto the second electrode surface immobilizing the second probe molecule within the matrix. This process is repeated until the array of electrodes is completed. Each electrode of the array will have a different probe molecule embedded within the conductive polymer matrix. Alternatively, the substrate with individually addressable electrodes is exposed to a monomer solution, and all of the electrodes are activated to electrochemically polymerize the conductive polymer onto the surfaces of the electrodes. The substrate is then thoroughly rinsed and probe molecules are covalently attached to the surface of the conductive polymer at each electrode by spotting technologies well known in the art. In this manner production of the array is simplified by only requiring one electrochemical polymerization and subsequent rinsing. The density of the array is only limited by the number of individually addressable electrodes that can be placed on a substrate. Additionally, the electrodes cannot be so close together so as to permit the conductive polymer matrix of one electrode to contact that of an adjacent matrix, i.e., to prevent cross-talk.

The solid substrate can be made of a wide variety of materials and can be configured in a large number of ways, as is discussed herein and will be apparent to one of skill in the art. In addition, a single device may comprise more than one substrate; for example, there may be a “sample treatment” cassette that interfaces with a separate “detection” cassette; a raw sample is added to the sample treatment cassette and is manipulated to prepare the sample for detection, which is removed from the sample treatment cassette and added to the detection cassette. There may be an additional functional cassette into which the device fits; for example, a heating element which is placed in contact with the sample cassette to effect reactions such as PCR. In some cases, a portion of the substrate may be removable; for example, the sample cassette may have a detachable detection cassette, such that the entire sample cassette is not contacted with the detection apparatus. See for example U.S. Pat. No. 5,603,351, PCT US96/17116, and “Multilayered Microfluidic Devices for Analyte Reactions” filed in the PCT Dec. 11, 2000, Serial No. PCT/US00/33499, hereby incorporated by reference.

The composition of the solid substrate will depend on a variety of factors, including the techniques used to create the device, the use of the device, the composition of the sample, the analyte to be detected, the size of the wells and microchannels, the presence or absence of electronic components, etc. Generally, the devices of the invention should be easily sterilizable as well. The supporting substrate of the apparatus of the invention is advantageously made from any solid material, including but not limited to silicon such as silicon wafers, silicon dioxide, silicon nitride, glass and fused silica, gallium arsenide, indium phosphide, aluminum, ceramics, polyimide, quartz, plastics, resins and polymers including polymethylmethacrylate, acrylics, polyethylene, polyethylene terepthalate, polycarbonate, polystyrene and other styrene copolymers, polypropylene, polytetrafluoroethylene, superalloys, zircaloy, steel, gold, silver, copper, tungsten, molybdeumn, tantalum, Kovar™, Kevlar™, Kapton™, Mylar™, brass, sapphire, etc. High melting borosilicate or fused silicas may be preferred for their UV transmission properties when any of the sample manipulation steps require light based technologies. In addition, as outlined herein, portions of the internal surfaces of the device may be coated with a variety of coatings as needed, to reduce non-specific binding, to allow the attachment of binding ligands, for biocompatibility, for flow resistance, etc. In preferred embodiments, the supporting substrate of the apparatus of the present invention is composed of silicon or glass. The linker moieties are embedded within or placed in contact with the supporting substrate.

In another preferred embodiment, the substrate is made from printed circuit board (PCP) materials, including without limitation, fiberglass, teflon, ceramics, glass, silicon, mica, plastic (including acrylics, polystyrene and copolymers of styrene and other materials, polypropylene, polyethylene, polybutylene, polycarbonate, polyurethanes, Teflon™, and derivatives thereof, etc.), GETEK (a blend of polypropylene oxide and fiberglass), etc.

The supporting substrate has a surface area of from about 0.01 μm² to about 5 cm² containing between 1 and 1×10⁸ microelectrodes in contact with said linker moieties. In a preferred embodiment, the supporting substrate has a surface area of 10,000 μm² and contains 10⁴ microelectrodes in contact with linker moieties. In preferred embodiments, the microelectrodes are arranged on the supporting substrate so that they are separated by a distance of from about 0.05 μm to 0.5 mm. In more preferred embodiments, the microelectrodes are regularly spaced on the solid supporting substrate with a uniform spacing there between.

In general, circuit board materials are those that comprise an insulating substrate that is coated with a conducting layer and processed using lithography techniques, particularly photolithography techniques, to form the patterns of electrodes and interconnects (sometimes referred to in the art as interconnections or leads). The insulating substrate is generally, but not always, a polymer. As is known in the art, one or a plurality of layers may be used, to make either “two dimensional” (e.g. all electrodes and interconnections in a plane) or “three dimensional” (wherein the electrodes are on one surface and the interconnects may go through the board to the other side) boards. Three dimensional systems frequently rely on the use of drilling or etching, followed by electroplating with a metal such as copper, such that the “through board” interconnections are made. Circuit board materials are often provided with a foil already attached to the substrate, such as a copper foil, with additional copper added as needed (for example for interconnections), for example by electroplating. The copper surface may then need to be roughened, for example through etching, to allow attachment of the adhesion layer.

In an alternative embodiment the solid substrate comprises ceramic materials, such as are outlined in U.S. Ser. Nos. 09/235,081; 09/337,086; 09/464,490; 09/492,013; 09/466,325; 09/460,281; 09/460,283; 09/387,691; 09/438,600; 09/506,178; and 09/458,534; all of which are expressly incorporated by reference in their entirety. In this embodiment, the devices are made from layers of green-sheet that have been laminated and sintered together to form a substantially monolithic structure. Green-sheet is a composite material that includes inorganic particles of glass, glass-ceramic, ceramic, or mixtures thereof, dispersed in a polymer binder, and may also include additives such as plasticizers and dispersants. The green-sheet is preferably in the form of sheets that are 50 to 250 microns thick. The ceramic particles are typically metal oxides, such as aluminum oxide or zirconium oxide. An example of such a green-sheet that includes glass-ceramic particles is “AX951” that is sold by E. I. Du Pont de Nemours and Company. An example of a green-sheet that includes aluminum oxide particles is “Ferro Alumina” that is sold by Ferro Corp. The composition of the green-sheet may also be custom formulated to meet particular applications. The green-sheet layers are laminated together and then fired to form a substantially monolithic multilayered structure. The manufacturing, processing, and applications of ceramic green-sheets are described generally in Richard E. Mistler, “Tape Casting: The Basic Process for Meeting the Needs of the Electronics Industry,” Ceramic Bulletin, 69(6):1022-1026 (1990), and in U.S. Pat. No. 3,991,029, which are incorporated herein by reference.

The method for fabricating devices begins with providing sheets of green-sheet that are preferably 50 to 250 microns thick. The sheets of green-sheet are cut to the desired size, typically 6 inches by 6 inches for conventional processing, although smaller or larger devices may be used as needed. Each green-sheet layer may then be textured using various techniques to form desired structures, such as vias, channels, or cavities, in the finished multilayered structure. In preferred embodiments an array of cavities or wells are constructed. In this preferred embodiment, each well has a micro-electrode in electrical contact with a linking moiety (preferably polyacrylamide gel), wherein the binding molecule is bound to the linking moiety.

Various techniques may be used to texture a green-sheet layer. For example, portions of a green-sheet layer may be punched out to form vias or channels. This operation may be accomplished using conventional multilayer ceramic punches, such as the Pacific Trinetics Corp. Model APS-8718 Automated Punch System. Instead of punching out part of the material, features, such as channels and wells may be embossed into the surface of the green-sheet by pressing the green-sheet against an embossing plate that has a negative image of the desired structure. Texturing may also be accomplished by laser tooling with a laser via system, such as the Pacific Trinetics LVS-3012. Next, a wide variety of materials may be applied, preferably in the form of thick-film pastes, to each textured green-sheet layer. For example, electrically conductive pathways may be provided by depositing metal-containing thick-film pastes onto the green-sheet layers. Thick-film pastes typically include the desired material, which may be either a metal or a dielectric, in the form of a powder dispersed in an organic vehicle, and the pastes are designed to have the viscosity appropriate for the desired deposition technique, such as screen-printing. The organic vehicle may include resins, solvents, surfactants, and flow-control agents. The thick-film paste may also include a small amount of a flux, such as a glass frit, to facilitate sintering. Thick-film technology is further described in J. D. Provance, “Performance Review of Thick Film Materials,” Insulation/Circuits (April, 1977) and in Morton L. Topfer, Thick Film Microelectronics, Fabrication, Design, and Applications (1977), pp. 41-59, which are incorporated herein by reference.

The porosity of the resulting thick-film can be adjusted by adjusting the amount of organic vehicle present in the thick-film paste. Specifically, the porosity of the thick-film can be increased by increasing the percentage of organic vehicle in the thick-film paste. Similarly, the porosity of a green-sheet layer can be increased by increasing the proportion of organic binder. Another way of increasing porosity in thick-films and green-sheet layers is to disperse within the organic vehicle, or the organic binder, another organic phase that is not soluble in the organic vehicle. Polymer microspheres can be used advantageously for this purpose.

To add electrically conductive pathways, the thick film pastes typically include metal particles, such as silver, platinum, palladium, gold, copper, tungsten, nickel, tin, or alloys thereof. Silver pastes are preferred. Examples of suitable silver pastes are silver conductor composition numbers 7025 and 7713 sold by E. I. Du Pont de Nemours and Company.

The thick-film pastes are preferably applied to a green-sheet layer by screen-printing. In the screen-printing process, the thick-film paste is forced through a patterned silk screen so as to be deposited onto the green-sheet layer in a corresponding pattern. Typically, the silk screen pattern is created photographically by exposure to a mask. In this way, conductive traces may be applied to a surface of a green-sheet layer. Vias present in the green-sheet layer may also be filled with thick-film pastes. If filled with thick-filled pastes containing electrically conductive materials, the vias can serve to provide electrical connections between layers and/or into wells within an array of wells.

After the desired structures are formed in each layer of green-sheet, preferably a layer of adhesive is applied to either surface of the green-sheet. Preferably, the adhesive is a room-temperature adhesive. Such room-temperature adhesives have glass transition temperatures below room temperature, i.e., below about 20° C., so that they can bind substrates together at room temperature. Moreover, rather than undergoing a chemical change or chemically reacting with or dissolving components of the substrates, such room-temperature adhesives bind substrates together by penetrating into the surfaces of the substrates. Sometimes such room-temperature adhesives are referred to as “pressure-sensitive adhesives.” Suitable room-temperature adhesives are typically supplied as water-based emulsions and are available from Rohm and Haas, Inc. and from Air Products, Inc. For example, a material sold by Air Products, Inc. as “Flexcryl 1653” has been found to work well.

The room-temperature adhesive may be applied to the green-sheet by conventional coating techniques. To facilitate coating, it is often desirable to dilute the supplied pressure-sensitive adhesive in water, depending on the coating technique used, and on the viscosity and solids loading of the starting material. After coating, the room-temperature adhesive is allowed to dry. The dried thickness of the film of room-temperature adhesive is preferably in the range of 1 to 10 microns, and the thickness should be uniform over the entire surface of the green-sheet. Film thicknesses that exceed 15 microns are undesirable. With such thick films of adhesive voiding or delamination can occur during firing due to the large quantity of organic material that must be removed. Films that are less than about 0.5 microns thick when dried are too thin, because they provide insufficient adhesion between the layers of green-sheet.

From among conventional coating techniques, spin-coating and spraying are the preferred methods. If spin-coating is used, it is preferable to add 1 gram of deionized water for every 10 grams of “Flexcryl 1653.” If spraying is used, a higher dilution level is preferred to facilitate ease of spraying. Additionally, when room-temperature adhesive is sprayed on, it is preferable to hold the green-sheet at an elevated temperature, e.g., about 60 to 70° C., so that the material dries nearly instantaneously as it is deposited onto the green-sheet. The instantaneous drying results in a more uniform and homogeneous film of adhesive.

After the room-temperature adhesive has been applied to the green-sheet layers, the layers are stacked together to form a multilayered green-sheet structure. Preferably, the layers are stacked in an alignment die, so as to maintain the desired registration between the structures of each layer. When an alignment die is used, alignment holes must be added to each green-sheet layer. Typically, the stacking process alone is sufficient to bind the green-sheet layers together when a room-temperature adhesive is used. In other words, little or no pressure is required to bind the layers together. However, in order to effect a more secure binding of the layers, the layers are preferably laminated together after they are stacked.

The lamination process involves the application of pressure to the stacked layers. For example, in the conventional lamination process, a uniaxial pressure of about 1000 to 1500 psi is applied to the stacked green-sheet layers that is then followed by an application of an isostatic pressure of about 3000 to 5000 psi for about 10 to 15 minutes at an elevated temperature, such as 70_C. Adhesives do not need to be applied to bind the green-sheet layers together when the conventional lamination process is used.

However, pressures less than 2500 psi are preferable in order to achieve good control over the dimensions of such structures as internal or external cavities and channels. Even lower pressures are more desirable to allow the formation of larger structures, such as cavities and channels. For example, if a lamination pressure of 2500 psi is used, the size of well-formed internal cavities and channels is typically limited to no larger than roughly 20 microns. Accordingly, pressures less than 1000 psi are more preferred, as such pressures generally enable structures having sizes greater than about 100 microns to be formed with some measure of dimensional control. Pressures of less than 300 psi are even more preferred, as such pressures typically allow structures with sizes greater than 250 microns to be formed with some degree of dimensional control. Pressures less than 100 psi, which are referred to herein as “near-zero pressures,” are most preferred, because at such pressures, few limits exist on the size of internal and external cavities and channels that can be formed in the multilayered structure.

The pressure is preferably applied in the lamination process by means of a uniaxial press. Alternatively, pressures less than about 100 psi may be applied by hand. As with semiconductor device fabrication, many devices may be present on each sheet. Accordingly, after lamination the multilayered structure may be diced using conventional green-sheet dicing or sawing apparatus to separate the individual devices. The high level of peel and shear resistance provided by the room-temperature adhesive results in the occurrence of very little edge delamination during the dicing process. If some layers become separated around the edges after dicing, the layers may be easily re-laminated by applying pressure to the affected edges, for example by hand, without adversely affecting the rest of the device.

The final processing step is firing to convert the laminated multilayered green-sheet structure from its “green” state to form the finished, substantially monolithic, multilayered structure. The firing process occurs in two important stages as the temperature is raised. The first important stage is the binder burnout stage that occurs in the temperature range of about 250 to 500° C., during which the other organic materials, such as the binder in the green-sheet layers and the organic components in any applied thick-film pastes, are removed from the structure.

The sintering stage, the next important stage of firing, occurs at a higher temperature, in which the inorganic particles sinter together. Sintering results in the multilayered becoming densified and substantially monolithic. The sintering temperature used depends on the nature of the inorganic particles present in the green-sheet. For many types of ceramics, appropriate sintering temperatures range from about 950 to about 1600° C., depending on the material. For example, for green-sheet containing aluminum oxide, sintering temperatures between 1400 and 1600° C. are typical. Other ceramic materials, such as silicon nitride, aluminum nitride, and silicon carbide require higher sintering temperatures, namely 1700 to 2200° C. For green-sheet with glass-ceramic particles, a sintering temperature in the range of 750 to 950° C. is typical. Glass particles generally require sintering temperatures in the range of only about 350 to 700° C. Finally, metal particles may require sintering temperatures anywhere from 550 to 1700° C., depending on the metal.

Typically, the devices are fired for a period of about 4 hours to about 12 hours or more, depending on the material used. Generally, the firing should be of a sufficient duration so as to remove the organic materials from the structure, and to completely sinter the inorganic particles. In particular, polymers are present as a binder in the green-sheet and in the room-temperature adhesive, and the firing should be of sufficient temperature and duration to decompose these polymers, and to allow for their removal from the multilayered structure.

Typically, the multilayered structure undergoes a reduction in volume during the firing process. During the binder burnout phase, a small volume reduction of about 0.5 to 1.5% is normally observed. At higher temperatures, during the sintering stage, a further volume reduction of about 14 to 17% is typically observed.

The volume change due to firing, on the other hand, can be controlled. In particular, to match volume changes in two materials, such as green-sheet and thick-film paste, one should match: (1) the particle sizes; and (2) the percentage of organic components, such as binders, which are removed during the firing process. Volume changes need not be matched exactly, but any mismatch will typically result in internal stresses in the device. Additionally, symmetrical processing, placing the identical material or structure on opposite sides of the device can, to some extent, compensate for shrinkage stresses caused by mismatched materials. Too great a mismatch in either sintering temperatures or volume changes may result in defects in or failure of some or all of the device. For example, the device may delaminate into its individual layers, or it may become warped or distorted. Dissimilar materials, such as thick-film pastes or other green-sheet layers, may be added prior to or after the firing process. As noted above, preferably any dissimilar materials added to the green-sheet layers prior to firing, and the dissimilar materials and the green-sheet layers are co-fired. The benefit of co-firing is that the added materials are sintered to the green-sheet layers and become integral to the substantially monolithic microfluidic device. However, to be co-fireable, the added materials should have sintering temperatures and volume changes due to firing that are matched with those of the green-sheet layers. Sintering temperatures are largely material-dependent, so that matching sintering temperatures simply requires proper selection of materials. For example, although silver is the preferred metal for providing electrically conductive pathways, if the green-sheet layers contain alumina particles, which require a sintering temperature in the range of 1400 to 1600° C., some other metal, such as platinum, must be used due to the relatively low melting point of silver (961° C.). Alternatively, the addition of other substrates or joining of two post-sintered ( i.e. post fired) pieces can be done using any variety of adhesive techniques, including those outlined herein. For example, two “halves” of a device can be glued or fused together. Thus, in a preferred embodiment polyacrylamide gel and biological components, which are not stable at high temperature, can be sandwiched in between the two halves. Alternatively, ceramic devices comprising open channels or wells can be made, additional substrates or materials placed into the devices, and then they may be sealed with other materials.

The substrates of the invention can form microfluidic cassettes or devices that can be used to effect a number of manipulations on a sample to ultimately result in cell detection or quantification. These manipulations can include cell handling (cell concentration, cell lysis, cell removal, cell separation, etc.), separation of the desired cell from other sample components, chemical or enzymatic reactions on the cell, detection of the cells or other components, etc. The devices of the invention can include one or more wells for sample manipulation, waste or reagents; microchannels to and between these wells, including microchannels containing electrophoretic separation matrices; valves to control fluid movement; on-chip pumps such as electroosmotic, electrohydrodynamic, or electrokinetic pumps; and detection systems. The devices of the invention can be configured to manipulate one or multiple samples or analytes.

The devices of the invention can be made in a variety of ways, as will be appreciated by those in the art. See for example WO96/39260, directed to the formation of fluid-tight electrical conduits; U.S. Pat. No. 5,747,169, directed to sealing; EP 0637996 B1; EP 0637998 B1; WO96/39260; WO97/16835; WO98/13683; WO97/16561; WO97/43629; WO96/39252; WO96/15576; WO96/15450; WO97/37755; and WO97/27324; and U.S. Pat. Nos. 5,304,487; 5,071531; 5,061,336; 5,747,169; 5,296,375; 5,110,745; 5,587,128; 5,498,392; 5,643,738; 5,750,015; 5,726,026; 5,35,358; 5,126,022; 5,770,029; 5,631,337; 5,569,364; 5,135,627; 5,632,876; 5,593,838; 5,585,069; 5,637,469; 5,486,335; 5,755,942; 5,681,484; and 5,603,351, all of which are hereby incorporated by reference. Suitable fabrication techniques again will depend on the choice of substrate, but preferred methods include, but are not limited to, a variety of micromachining and microfabrication techniques, including film deposition processes such as spin coating, chemical vapor deposition, laser fabrication, photolithographic and other etching techniques using either wet chemical processes or plasma processes, embossing, injection molding and bonding techniques (see U.S. Pat. No. 5,747,169, hereby incorporated by reference). In addition, there are printing techniques for the creation of desired fluid guiding pathways; that is, patterns of printed material can permit directional fluid transport. Thus, the build-up of “ink” can serve to define a flow channel. In addition, the use of different “inks” or “pastes” can allow different portions of the pathways having different flow properties. For example, materials can be used to change solute/solvent RF values (the ratio of the distance moved by a particular solute to that moved by a solvent front). For example, printed fluid guiding pathways can be manufactured with a printed layer or layers comprised of two different materials, providing different rates of fluid transport. Multi-material fluid guiding pathways can be used when it is desirable to modify retention times of reagents in fluid guiding pathways. Furthermore, printed fluid guiding pathways can also provide regions containing reagent substances, by including the reagents in the “inks” or by a subsequent printing step. See for example U.S. Pat. No. 5,795,453, herein incorporated by reference in its entirety.

In addition, it should be understood that while most of the discussion herein is directed to the use of planar substrates with microchannels and wells, other geometries can be used as well. For example, two or more planar substrates can be stacked to produce a three dimensional device, that can contain microchannels flowing within one plane or between planes; similarly, wells may span two or more substrates to allow for larger sample volumes. Thus for example, both sides of a substrate can be etched to contain microchannels; see for example U.S. Pat. Nos. 5,603,351 and 5,681,484, both of which are hereby incorporated by reference.

Thus, the devices of the invention may include at least one microchannel or flow channel that allows the flow of sample from the sample inlet port to the other components or modules of the system. The collection of microchannels and wells is sometimes referred to in the art as a “mesoscale flow system”. As will be appreciated by those in the art, the flow channels may be configured in a wide variety of ways, depending on the use of the channel. For example, a single flow channel starting at the sample inlet port may be separated into a variety of smaller channels, such that the original sample is divided into discrete subsamples for parallel processing or analysis. Alternatively, several flow channels from different modules, for example the sample inlet port and a reagent storage module may feed together into a mixing chamber or a reaction chamber. As will be appreciated by those in the art, there are a large number of possible configurations; what is important is that the flow channels allow the movement of sample and reagents from one part of the device to another. For example, the path lengths of the flow channels may be altered as needed; for example, when mixing and timed reactions are required, longer and sometimes tortuous flow channels can be used.

In some embodiments, the devices herein are designed on a scale suitable to analyze microvolumes, although in some embodiments large samples (e.g. cc's of sample) may be reduced in the device to a small volume for subsequent analysis. That is, “mesoscale” as used herein refers to chambers and microchannels that have cross-sectional dimensions on the order of 0.1 μm to 500 μm. The mesoscale flow channels and wells have preferred depths on the order of 0.1 μm to 100 μm, typically 2-50 μm. The channels have preferred widths on the order of 2.0 to 500 μm, more preferably 3-100 μm. For many applications, channels of 5-50 μm are useful. However, for many applications, larger dimensions on the scale of millimeters may be used. Similarly, chambers (sometimes also referred to herein as “wells”) in the substrates often will have larger dimensions, on the scale of a few millimeters. In addition to the flow channel system, the devices of the invention are configured to include one or more of a variety of components, herein referred to as “modules”, that will be present on any given device depending on its use. These modules include, but are not limited to: sample inlet ports; sample introduction or collection modules; cell handling modules (for example, for cell lysis, cell removal, cell concentration, cell separation or capture, cell growth, etc.); separation modules, for example, for electrophoresis, dielectrophoresis, gel filtration, ion exchange/affinity chromatography (capture and release) etc.; reaction modules for chemical or biological alteration of the sample, including amplification of the nucleic acids, chemical, physical or enzymatic cleavage or alteration of the sample components, or chemical modification of the target; fluid pumps; fluid valves; thermal modules for heating and cooling; storage modules for assay reagents; mixing chambers; and detection modules.

In a preferred embodiment, the devices of the invention include at least one sample inlet port for the introduction of the sample to the device. This may be part of or separate from a sample introduction or collection module; that is, the sample may be directly fed in from the sample inlet port to a separation chamber, or it may be pretreated in a sample collection well or chamber.

In a preferred embodiment, the devices of the invention include a sample collection module, which can be used to concentrate or enrich the sample if required; for example, see U.S. Pat. No. 5,770,029, including the discussion of enrichment channels and enrichment means.

In a preferred embodiment, the devices of the invention include a cell handling module. Thus, for example, the detection of particular antibodies in blood can require the removal of the blood cells for efficient analysis, or the cells (and/or nucleus) may be lysed. In this context, “cells” include eukaryotic and prokaryotic cells, and viral particles that may require treatment prior to analysis, such as the release of nucleic acid from a viral particle prior to detection of target sequences. In addition, cell handling modules may also utilize a downstream means for determining the presence or absence of cells. Suitable cell handling modules include, but are not limited to, cell lysis modules, cell removal modules, cell concentration modules, and cell separation or capture modules. In addition, as for all the modules of the invention, the cell handling module is in fluid communication via a flow channel with at least one other module of the invention.

In a preferred embodiment, the cell handling module includes a cell lysis module. As is known in the art, cells may be lysed in a variety of ways, depending on the cell type. In one embodiment, as described in EP 0 637 998 B1 and U.S. Pat. No. 5,635,358, hereby incorporated by reference, the cell lysis module may comprise cell membrane piercing protrusions that extend from a surface of the cell handling module. As fluid is forced through the device, the cells are ruptured. Similarly, this may be accomplished using sharp edged particles trapped within the cell handling region. Alternatively, the cell lysis module can comprise a region of restricted cross-sectional dimension, which results in cell lysis upon pressure.

In a preferred embodiment, the cell lysis module comprises a cell lysing agent, such as guanidium chloride, chaotropic salts, enzymes such as lysozymes, etc. In some embodiments, for example for blood cells, a simple dilution with water or buffer can result in hypotonic lysis. The lysis agent may be solution form, stored within the cell lysis module or in a storage module and pumped into the lysis module. Alternatively, the lysis agent may be in solid form, that is taken up in solution upon introduction of the sample.

The cell lysis module may also include, either internally or externally, a filtering module for the removal of cellular debris as needed. This filter may be microfabricated between the cell lysis module and the subsequent module to enable the removal of the lysed cell membrane and other cellular debris components; examples of suitable filters are shown in EP 0 637 998 B1, incorporated by reference.

In a preferred embodiment, the cell handling module includes a cell separation or capture module. This embodiment utilizes a cell capture region comprising binding sites capable of reversibly binding a cell surface molecule to enable the selective isolation (or removal) of a particular type of cell from the sample. These binding moieties may be immobilized either on the surface of the module or on a particle trapped within the module (i.e. a bead) by physical absorption or by covalent attachment. Suitable binding moieties will depend on the cell type to be isolated or removed, and generally includes antibodies and other binding ligands, such as ligands for cell surface receptors, etc. as outlined herein Thus, a particular cell type may be removed from a sample prior to further handling, or the assay is designed to specifically bind the desired cell type, wash away the non-desirable cell types, followed by either release of the bound cells by the addition of reagents or solvents, physical removal (i.e. higher flow rates or pressures), or even in situ lysis.

Alternatively, a cellular “sieve” can be used to separate cells on the basis of size. This can be done in a variety of ways, including protrusions from the surface that allow size exclusion, a series of narrowing channels, a weir, or a diafiltration type setup.

In a preferred embodiment, the cell handling module includes a cell removal module. This may be used when the sample contains cells that are not required in the assay or are undesirable. Generally, cell removal will be done on the basis of size exclusion as for “sieving”, above, with channels exiting the cell handling module that are too small for the cells.

In a preferred embodiment, the cell handling module includes a cell concentration module. As will be appreciated by those in the art, this is done using “sieving” methods, for example to concentrate the cells from a large volume of sample fluid prior to lysis.

In some embodiments of the present invention, the microelectrodes project from the surface of the substrate, with such projections extending between 5×10⁻⁸ and 1×10⁻⁵ cm from the surface of the supporting substrate. In other embodiments, the microelectrodes comprise a flat disk of conductive material that is embedded in the supporting substrate and is exposed at the substrate surface, with the supporting substrate acting as an insulator in the spaces between the microelectrodes.

In a preferred embodiment of the present invention the microelectrodes comprise a gold or platinum conductor and a glass or silicon insulator. In alternative embodiments, the microelectrodes comprise conductor substances such as solid or porous, foils or films of silver, titanium, or copper, or metal oxides, metal nitrides, metal carbides, carbon, graphite, or combinations thereof. In additional embodiments, the microelectrodes comprise substrate and/or insulator substances such as plastic, rubber, fabric, ceramics, or combinations thereof. The microelectrodes of the present invention preferably have an exposed conductive surface of from about 0.01 μm² to 0.5 cm². In a preferred embodiment, the exposed conductive material has an area of from about 100 to 160,000 μm².

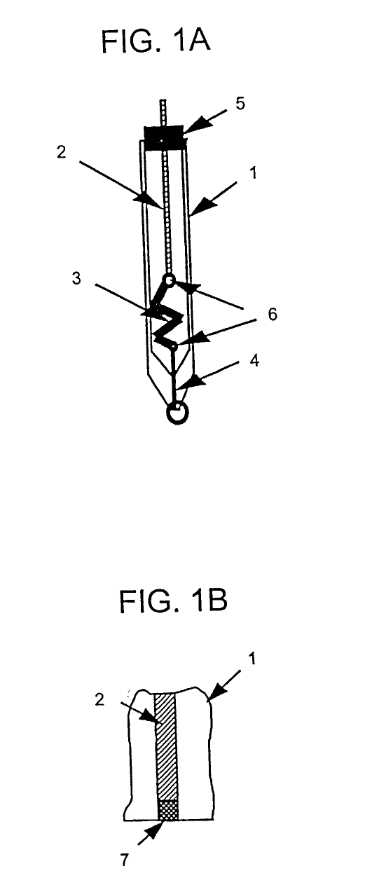

One embodiment of the present invention is shown in FIG. 1A, wherein the microelectrode comprises a glass capillary tube 1, containing an ultra fine platinum wire 2, to which a transition wire 3 has been soldered 6. The transition wire 3 is soldered 6 in turn to a hookup wire 4, which protrudes from an epoxy plug 5 that seals the capillary tube. Polyacrylamide gel material 7 is packed into a recess etched into the exposed surface of the platinum wire 2. The polymeric hydrogel pad is preferably at least about 0.1 to 30 μm thick, more preferably at least about 0.5 to 10 μm thick, and most preferably about 0.5 μm thick.

The apparatus of the present invention comprises at least one counter-electrode. In a preferred embodiment of the present invention, the counter-electrode comprises a conductive material, with an exposed surface that is significantly larger than that of the individual microelectrodes. In a preferred embodiment, the counter electrode comprises platinum wire. In alternative embodiments, the counter electrode comprises solid or porous films of gold, silver, platinum, titanium, or copper, or metal oxides, metal nitrides, metal carbides, carbon, graphite, or combinations thereof.

In other embodiments of the present invention, the apparatus comprises at least one reference electrode. The reference electrode is particularly useful in assays for detecting the change in electrical potential after probe and target molecules are allowed to interact and then determining the number or concentration of bacterial cells in a sample using quantitative methods (e.g., voltammetry). In preferred embodiments, the reference electrode comprises a silver/silver chloride electrode. In alternative embodiments, the reference electrode comprises solid or porous films of gold, platinum, titanium, or copper, or metal oxides, metal nitrides, metal carbides, carbon, graphite, or combinations thereof.

In still further embodiments of the present invention, the apparatus further comprises a plurality of wells each of which encompasses at least one microelectrode in contact with a linker moiety and at least one counter-electrode. The term “wells” is used herein in its conventional sense, to describe a portion of the supporting substrate in which the microelectrode and at least one counter-electrode are contained in a defined volume; said wells can protrude from the surface of the supporting substrate, or be embedded therein.

Electrochemical contact between each of the microelectrodes and the counter electrode and/or the reference electrode is advantageously provided using an electrolyte solution in contact with each of the microelectrodes comprising the apparatus of the invention. Electrolyte solutions useful in the apparatus and methods of the invention include any electrolyte solution at physiologically-relevant ionic strength (equivalent to about 0.15 M NaCl) and neutral pH. Examples of electrolyte solutions useful with the apparatus and methods of the invention include, but are not limited to, phosphate buffered saline, HEPES buffered solutions, and sodium bicarbonate buffered solutions. Said electrolyte solutions are in contact with each of the microelectrodes of the apparatus of the invention, the counter-electrode and the reference electrode if provided, thereby providing electrochemical contact between the electrodes.

In preferred embodiments of the present invention, molecular interactions between probe molecules immobilized on linker moieties, within in contact with microelectrodes and protein or peptide target molecules in a sample mixture are detected by detecting an electrical signal using AC impedance. In other embodiments, such molecular interactions are detected by detecting an electrical signal using an electrical detection method selected from the group consisting of impedance spectroscopy, cyclic voltammetry, AC voltammetry, pulse voltammetry, square wave voltammetry, AC voltammetry, hydrodynamic modulation voltammetry, conductance, potential step method, potentiometric measurements, amperometric measurements, current step method, other steady-state or transient measurement methods, and combinations thereof.

In one embodiment of the apparatus of the present invention, the means for producing electrical impedance at each microelectrode is accomplished using a Model 1260 Impedance/Gain-Phase Analyser with Model 1287 Electrochemical Interface (Solartron Inc., Houston, Tex.). Other electrical impedance measurement means include, but are not limited to, transient methods using AC signal perturbation superimposed upon a DC potential applied to an electrochemical cell such as AC bridge and AC voltammetry. The measurements can be conducted at a certain particular frequency that specifically produces electrical signal changes that are readily detected or otherwise determined to be advantageous. Such particular frequencies are advantageously determined by scanning frequencies to ascertain the frequency producing, for example, the largest difference in electrical signal. The means for detecting changes in impedance at each microelectrode as a result of antibody-bacterial cell binding, or any other probe-target interaction can be accomplished by using any of the above-described instruments and analytical methods.

In a preferred embodiment the linker moiety is a conductive polymer and the probe molecule is covalently immobilized within the polymer matrix, non-covalently immobilized within the polymer matrix or covalently attached to the surface of the polymer matrix. When performing lableless (lack of electrochemical label or reporter attached to the probe or target) detection it is preferred to measure AC impedance using a Model 1260 Impedance/Gain-Pahse Analyser with Model 1287 Electrochemical interface, any other impedance gain-phase analyzer, AC bridge, or AC oscillographic polarography of Heyrovsky. Array based biochips require multi-site detection. AC impedance detection is preferred to other methods (such as DC scanning methods like voltammetry) as it can be carried out at higher frequencies, in the MHz range as opposed to the second range of DC voltammetry.

In a preferred embodiment of the present invention the target molecule is a protein (for example and without limitation an antigen, receptor, or ligand), which is detected in a sample using microelectrodes in contact with linker moieties to which binding ligands capable of specifically binding to the target protein (antibodies, small molecules, proteins, peptides, polypeptides, or aptamers) have been immobilized. In one method of the invention, AC impedance is measured at a plurality of microelectrodes in contact with linker moieties to which a probe having specificity to the target molecule has been immobilized. The microelectrodes are then exposed to a sample mixture containing a sample mixture containing the target molecule, and changes in AC impedance resulting from molecular interactions between the target molecules and binding ligands are then detected at each of the microelectrodes.

Alternatively the target is detected using microelectrodes in contact with linker moieties in which the binding ligands are embedded. Preferably the linker moiety is a conductive polymer. The AC impedance of the electrode is measured before and after exposure to a sample and the change in impedance is used to determine whether the target molecule is present.

It is known that in an ion doping process ion intercalation occurs in a conjugative conductive polymer matrix during an electrochemical process. In the doping process, cation expulsion and anion insertion occur during conductive polymer oxidation; anion ejection and cation insertion during the reverse reduction process. For example, the ion doping process of PPY can be shown as the following:

Oxidation: −(PPY)ₙ−+mnA⁻_−[(PPY)^{m+}Aₘ⁻]ₙ−+mne⁻

Reduction: −(PPY)ₙ−+mne⁻+mnM⁺_−[Mₘ⁺(PPY)^{m}]ₙ−

Where A− and M+ represent cation and anion, respectively.

While not wishing to be bound by any particular theory, it is thought that the molecular interaction of a target molecule and a probe molecule immobilized in or on a conductive polymer matrix interferes with the ion interaction process resulting in an increase of the resistance. The present invention uses this Faraday resistance (electrochemical reaction resistance) change, which is a result of the ion doping process. The Faraday resistance appears as a change in impedance from a base line measurement to that after a molecular interaction has taken place. This is explained more thoroughly in the Examples given below. Again without wishing to be bound by any particular theory, the present invention uses ion intercalation in a conjugative conductive polymer resulting from a target/probe molecular interaction to detect the presence of a target molecule in a sample. It has been found that the ion intercalation resistance significantly increases, as shown in Examples below, after probe/target molecular interaction, such as antibody/antigen bindings. The detection is accomplished without labeling the target or probe molecules. It is again noted that this preferred embodiment does not use an electrochemical or other type of reporter attached to the target or probe molecules. This is quite attractive because it can significantly reduce the manufacturing cost and simplifies the detection process. In addition, some targets simply cannot be labeled. In traditional immunoassay methods a sandwich method is utilized to label the second antibody for binding detection. In some cases the antigens are too small to be accessed (for example and without limitation small molecules such as AZT) by the labeled second antibody through the targets' epitopes. In these cases, reporterless or labelless detection must be used.

In some embodiments electrochemical reporters are used to enhance the detection of target molecules. For example and without limitation, electrochemical reporters (“reporter group”) is attached to the target molecule. Reporters are electrochemically-distinctive redox moieties, or an electron transfer moiety that do not interfere with the molecular interaction to be detected. Electrochemically-labeled target molecules are electrochemically active, i.e.., they are capable of participating in oxidation/reduction reaction under conditions of applied voltage potential, and are compatible with the probes, target, and solution carrying the target. Electrochemically-labeled target molecules useful in the methods of the present invention may be prepared by labeling suitable target molecules with any reporter group having an electrochemically-distinctive property, most preferably a redox (oxidation/reduction) potential that can be distinguished from other components of the binding reaction, and that does not interfere with the molecular interaction to be detected.

As used herein “electron transfer moiety”, “ETM”, or grammatical equivalents thereof means a compound or group that is capable of reversibly, semi-reversibly, or irreversibly transferring one or more electrons. As used herein “electron donor moiety”, “electron acceptor moiety”, “electron transfer moieties” or grammatical equivalents thereof refers to molecules capable of electron transfer under certain conditions, for example oxidation-reduction reactions. It is to be understood that electron donor and acceptor capabilities are relative; that is, a molecule which can lose an electron under certain experimental conditions will be able to accept an electron under different experimental conditions. It is to be understood that the number of possible electron donor moieties and electron acceptor moieties is very large, and that one skilled in the art of electron transfer compounds will be able to utilize a number of compounds in the present invention. Preferred electron transfer moieties include, but are not limited to, transition metal complexes, organic electron transfer moieties, and electrodes.

In preferred embodiments target molecules are labeled with reporters comprising a transition metal complex or an organic redox couple. Transition metals include those whose atoms have a partial or complete d shell of electrons; elements having the atomic numbers 21-30, 39-48, 57-80 and the lanthanide series. Suitable transition metals for use in the invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinium (Pt), scandium (Sc), titanium (Ti), Vanadium (V), chromium (Cr), manganese (Mn), nickel (Ni), Molybdenum (Mo), technetium (Tc), tungsten (W), and iridium (Ir). That is, the first series of transition metals, the platinum metals (Ru, Rh, Pd, Os, Ir and Pt), along with Fe, Re, W, Mo and Tc, are preferred. Particularly preferred are ruthenium, rhenium, osmium, platinium, cobalt and iron.

The transition metals are complexed with a variety of ligands, generally depicted herein as “L”, to form suitable transition metal complexes, as is well known in the art. Suitable ligands fall into two categories: ligands which use nitrogen, oxygen, sulfur, carbon or phosphorus atoms (depending on the metal ion) as the coordination atoms (generally referred to in the literature as sigma (σ) donors) and organometallic ligands such as metallocene ligands (generally referred to in the literature as pi (π) donors, and depicted herein as Lₘ). Suitable nitrogen donating ligands are well known in the art and include, but are not limited to, NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam) and isocyanide. Substituted derivatives, including fused derivatives, may also be used. In some embodiments, porphyrins and substituted derivatives of the porphyrin family may be used. See, e.g., Comprehensive Coordination Chemistry, Ed. Wilkinson et al., Pergammon Press, 1987, Chapters 13.2 (pp73-98), 21.1 (pp. 813-898) and 21.3 (pp 915-957), all of which are hereby expressly incorporated by reference.

Suitable sigma donating ligands using carbon, oxygen, sulfur and phosphorus are known in the art. For example, suitable sigma carbon donors are found in Cotton and Wilkenson, Advanced Organic Chemistry, 5th Edition, John Wiley & Sons, 1988, hereby incorporated by reference; see page 38, for example. Similarly, suitable oxygen ligands include crown ethers, water and others known in the art. Phosphines and substituted phosphines are also suitable; see page 38 of Cotton and Wilkenson. The oxygen, sulfur, phosphorus and nitrogen-donating ligands are attached in such a manner as to allow the heteroatoms to serve as coordination atoms.

In a preferred embodiment, organometallic ligands are used as reporters. In addition to purely organic compounds for use as redox moieties, and various transition metal coordination complexes with δ-bonded organic ligand with donor atoms as heterocyclic or exocyclic substituents, there is available a wide variety of transition metal organometallic compounds with π-bonded organic ligands (see, e.g., Advanced Inorganic Chemistry, 5th Ed., Cotton & Wilkinson, John Wiley & Sons, 1988, chapter 26; Organometallics, A Concise Introduction, Elschenbroich et al., 2nd Ed., 1992; VCH; and Comprehensive Organometallic Chemistry II, A Review of the Literature 1982-1994, Abel et al. Ed., Vol. 7, chapters 7, 8, 10 & 11, Pergamon Press, all hereby expressly incorporated by reference). Such organometallic ligands include cyclic aromatic compounds such as the cyclopentadienide ion [C₅H₅(−1)] and various ring substituted and ring fused derivatives, such as the indenylide (−1) ion, that yield a class of bis(cyclopentadieyl)metal compounds, (i.e. the metallocenes). See, e.g., Robins et al., J. Am. Chem. Soc. 104:1882-1893 (1982); and Gassman et al., J. Am. Chem. Soc. 108:4228-4229 (1986), incorporated by reference. Of these, ferrocene [(C₅H₅)₂Fe] and its derivatives are prototypical examples which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. Metallocene derivatives of a variety of the first, second and third row transition metals are potential candidates as redox moieties. Other potentially suitable organometallic ligands include cyclic arenes such as benzene, to yield bis(arene)metal compounds and their ring substituted and ring fused derivatives, of which bis(benzene)chromium is a prototypical example, Other acyclic π-bonded ligands such as the allyl(−1) ion, or butadiene yield potentially suitable organometallic compounds, and all such ligands, in conjuction with other π-bonded and δ-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are potential candidate redox moieties.

In addition to transition metal complexes, other organic electron donors and acceptors may be used in the invention. These organic molecules include, but are not limited to, riboflavin, xanthene dyes, azine dyes, acridine orange, N,N′-dimethyl-2,7-diazapyrenium dichloride (DAP²⁺), methylviologen, ethidium bromide, quinones such as N,N′-dimethylanthra(2,1,9-def:6,5,10-d′e′f′)diisoquinoline dichloride (ADIQ²⁺); porphyrins ([meso-tetrakis(N-methyl-x-pyridinium)porphyrin tetrachloride], varlamine blue B hydrochloride, Bindschedler's green; 2,6-dichloroindophenol, 2,6-dibromophenolindophenol; Brilliant crest blue (3-amino-9-dimethyl-amino-10-methylphenoxyazine chloride), methylene blue; Nile blue A (aminoaphthodiethylaminophenoxazine sulfate), indigo-5,5′,7,7′-tetrasulfonic acid, indigo-5,5′,7-trisulfonic acid; phenosafranine, indigo-5-monosulfonic acid; safranine T; bis(dimethylglyoximato)-iron(II) chloride; induline scarlet, neutral red, anthracene, coronene, pyrene, 9-phenylanthracene, rubrene, binaphthyl, DPA, phenothiazene, fluoranthene, phenanthrene, chrysene, 1,8-diphenyl-1,3,5,7-octatetracene, naphthalene, acenaphthalene, perylene, TMPD and analogs and subsitituted derivatives of these compounds.

In other embodiments of the present invention, molecules are labeled with the following non-limiting examples of electrochemically-active moieties: 1,4-benzoquinone, ferrocene, tetracyanoquinodimethane, N,N,N′,N′-tetramethyl p-phenylenediamine, tetrathiafulvalene, 9-aminoacridine, acridine orange, aclarubicin, daunomycin, doxorubicin, pirarubicin, ethidium bromide, ethidium monoazide, chlortetracycline, tetracycline, minocycline, Hoechst 33258, Hoechst 33342, 7-aminoactinomycin D, Chromomycin A₃, mithramycin A, Vinblastine, Rifampicin, Os(bipyridine)₂(dipyridophenazine)₂⁺, Co(bipyridine)₃³⁺, or Fe-bleomycin.

The electrochemically-active moiety comprising the electrochemically active reporter-labeled molecule used in certain embodiments of the methods of the present invention is optionally linked to the antibody molecule through a linker, preferably having a length of from about 10 to-about 20 Angstroms. The linker can be an organic moiety such as a hydrocarbon chain (CH₂)ₙ, wherein n is an integer from 1 to about 20, or can comprise an ether; ester, carboxyamide, or thioether moiety, or a combination thereof. The linker can also be an inorganic moiety such as siloxane (O—Si—O). The length of the linker is selected so that the electrochemically-active moiety does not interfere with the molecular interaction to be detected.

“Competition assays” may be performed in accordance with an alternative embodiment of the present invention. In this embodiment a known amount of a competitor molecule labeled with an electrochemical reporter is exposed to an array of immobilized probe molecules, most preferably before a reaction mixture containing an unlabeled target molecule is exposed to the array. The amount of target molecule present in the reaction mixture is then determined indirectly by measuring the amount of labeled competitor molecule that is displaced from the array following the addition of the reaction mixture. “Sandwich assays”, in which a target binding molecule labeled with an electrochemical reporter is exposed to an array of immobilized probe molecules following exposure of the array to a reaction mixture, may be performed in accordance with another alternative embodiment of the present invention. The target binding molecule is selected for its ability to interact with the target molecule without interfering with the interaction between the target molecules and the immobilized probe molecules. The “sandwich” assay is particularly useful for detecting target molecules having higher molecular weights and results in an increase in experimental specificity and sensitivity.

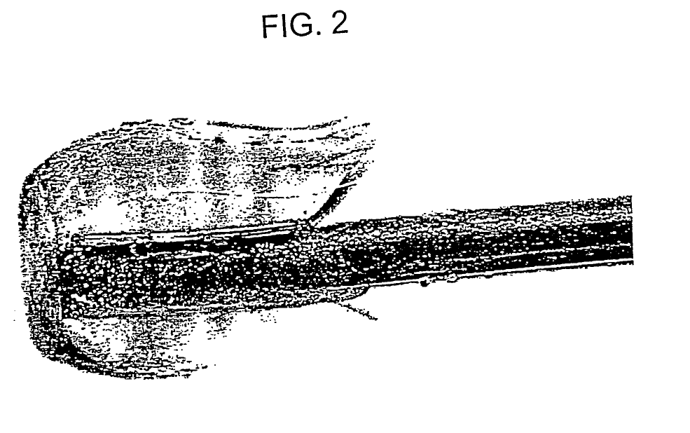

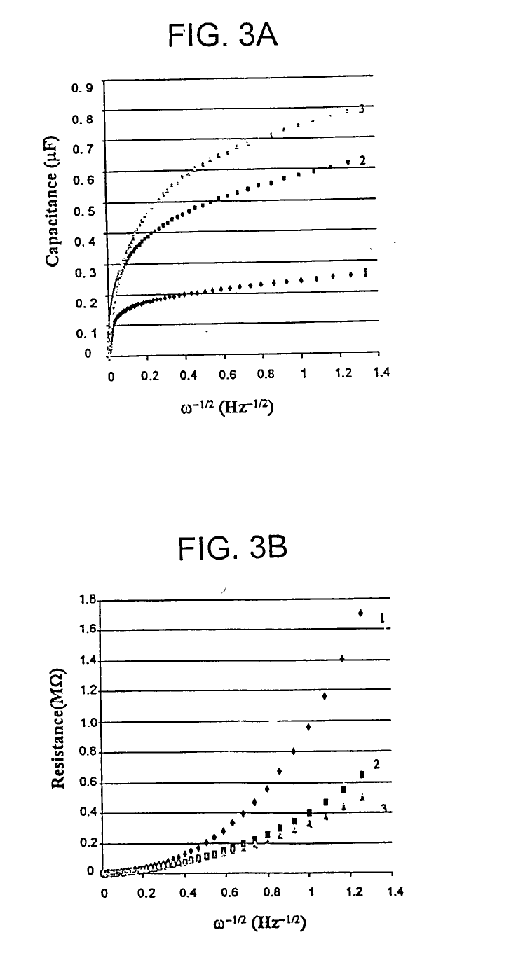

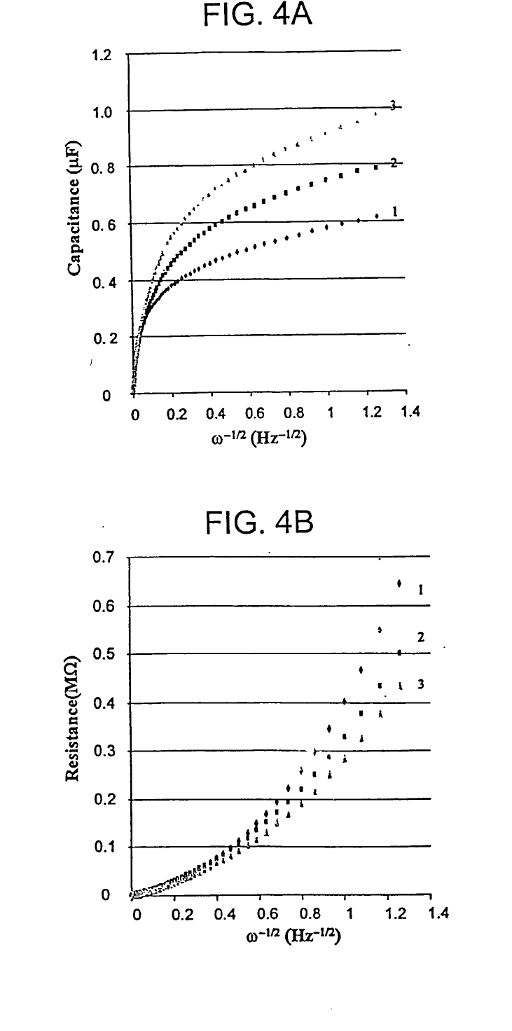

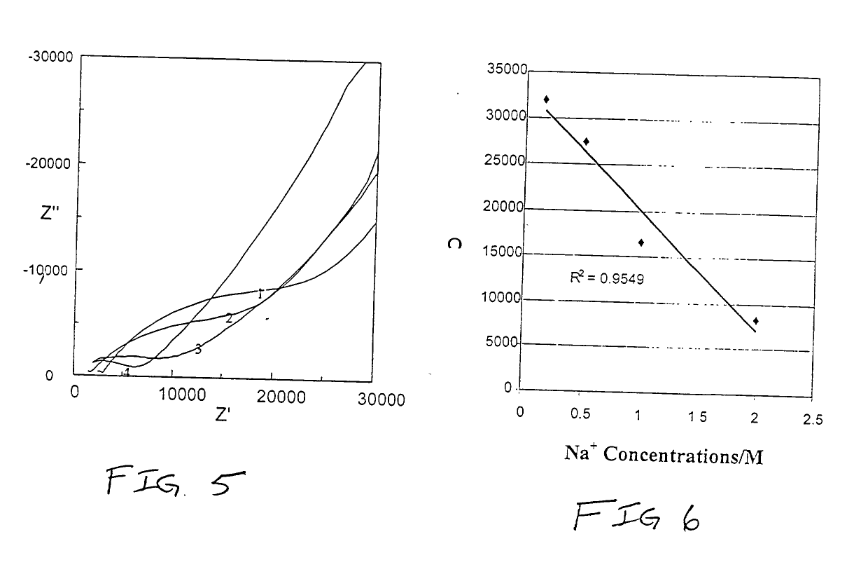

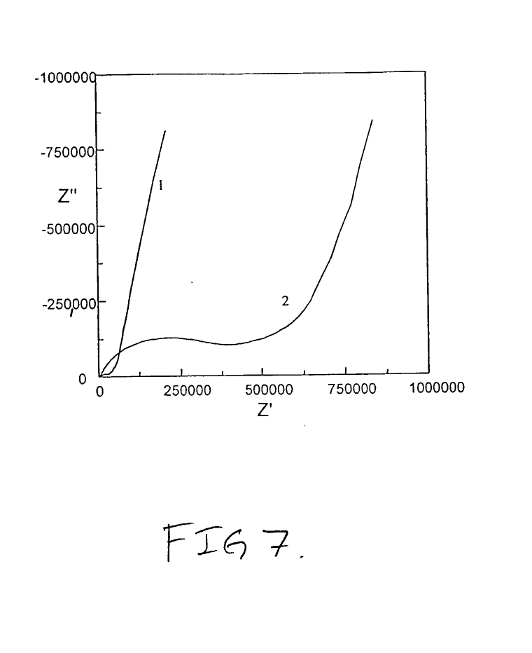

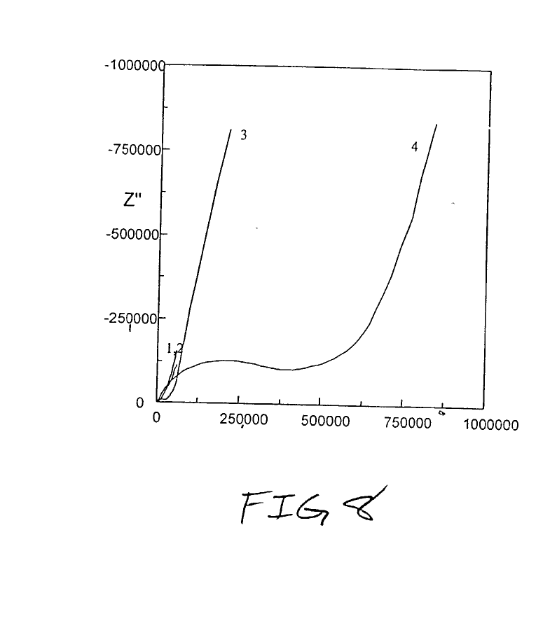

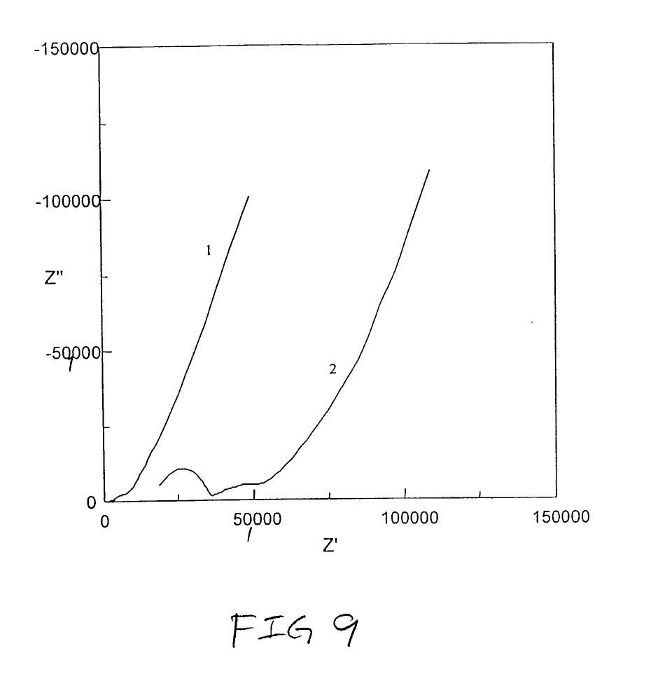

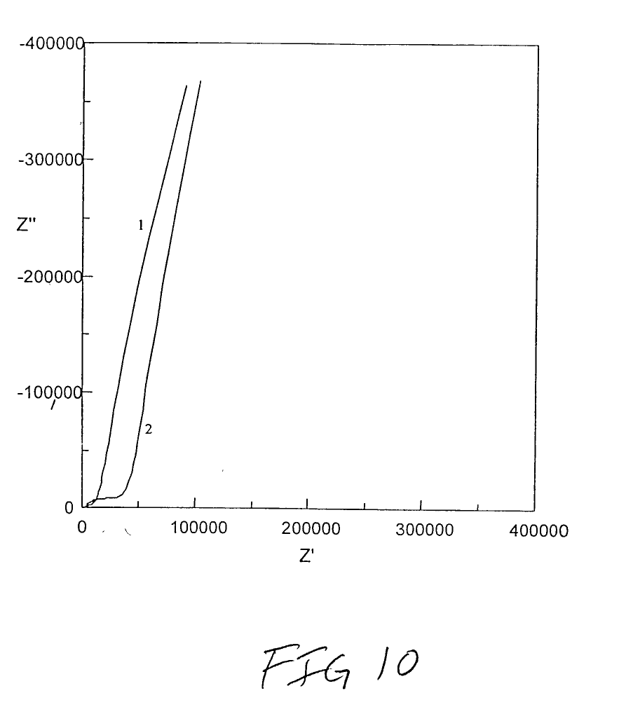

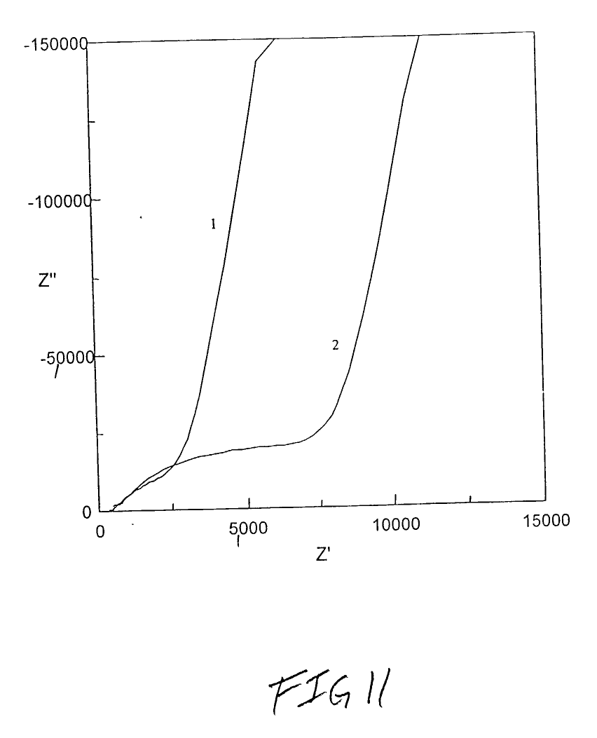

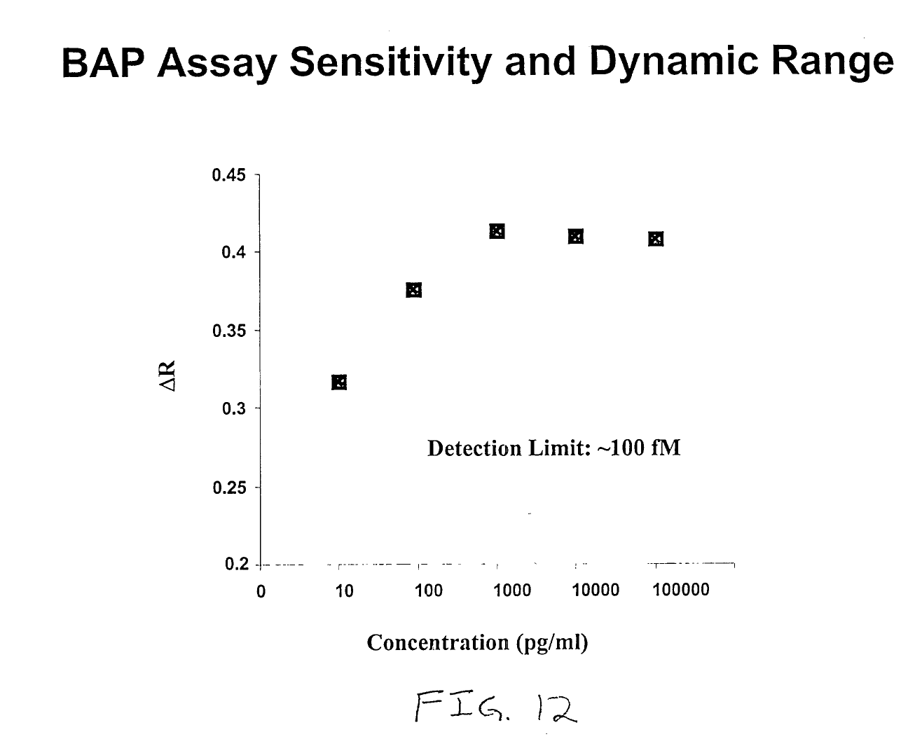

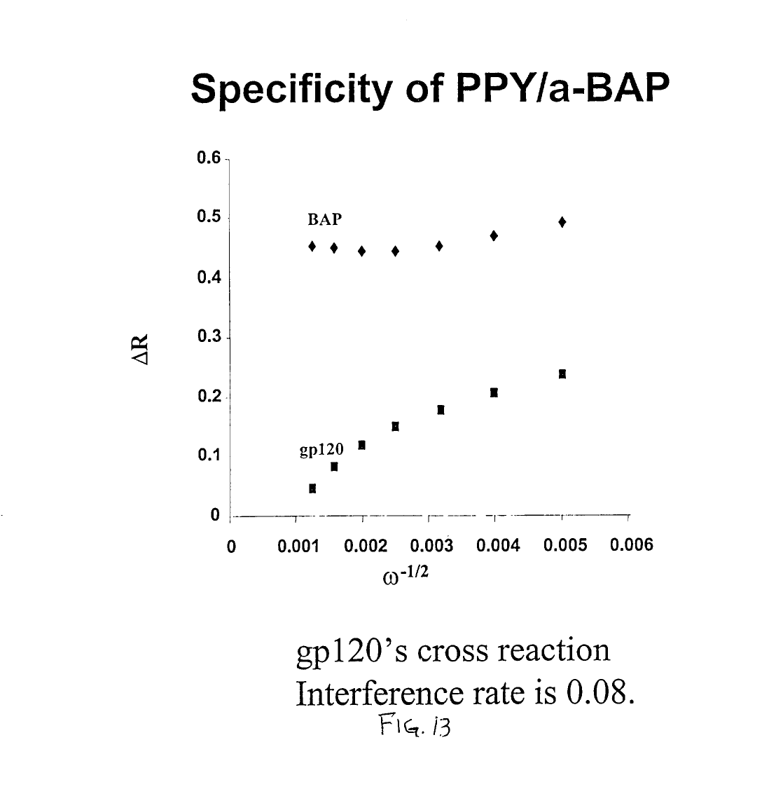

The preferred embodiments of the present invention are best understood by referring to FIGS. 1-6 and Examples 1-3. The Examples, which follow, are illustrative of specific embodiments of the invention, and various uses thereof. They are set forth for explanatory purposes only, and are not to be taken as limiting the invention.

For each of the examples described below a single microelectrode was constructed as depicted in FIG. 1. The radius of the microelectrode was approximately 50 μm.

### EXAMPLE 1

A solution containing 0.1 M pyrrole and PBS buffer was prepared as solution A. A solution for probe synthesis was prepared by adding 1 μL of 10 mg/mL unconjugated rabbit anti-BAP (Bacterial Alkaline Phosphatase) purchased from Biodesign into 99 μL of solution A.

The polypyrrole/protein probes layer was synthesized by an electrochemical method. The microelectrode was used as the working electrode. A platinum wire, with much larger surface area than that of the microelectrode, and a Ag/AgCl electrode were used as counter and reference electrodes, respectively. A CH 660 potentiostat was used to conduct cyclic voltammetry (CV) −0.1 to 1.0 vs. Ag/AgCl for four cycles to electrochemcally synthesize a polypyrrole/protein probe film onto the microelectrode surface. After the polypyrrole/protein probe film deposited on the microelectrode surface, a Solartron Impedance Frequency Analyzer 1260 with Electrochemical Interface 1287 was used to measure the impedance of the microelectrode in PBS buffer. A 2-electrode measurement system was used. The counter electrode was platinum wire, the surface area of which was much larger than the microelectrode.

PBS buffers with different concentrations of Na⁺ were prepared by adding different amounts of NaCl into 1× PBS buffer. AC impedance responses of the microelectrode with polypyrrole/protein probe film in PBS buffers with different concentrations Na⁺ were measured. FIG. 5 shows the results of electrochemical impedance spectroscopy, known in the art as a Nyquist Diagram, which plots the imaginary part of impedance (Z′) versus the real part measured at different frequences. In FIG. 5 curves 1-4 correspond to concentrations of 0.1, 0.5, 1, and 2 M Na⁺. In the high frequency portion of the curves semi-circles were observed; it is well known that the diameter of the semicircles represent Faraday resistance produced from electrochemical reactions. In electrochemcial impedance measurements, it relatively difficult to observe a complete semi-circle in the Nyquist Diagram since diffusion process frequencies mix with kinetic control process frequencies at relatively low frequencies. However, as is well known to the skilled artisan, the electrochemical reaction resistance can be calculated by extrapolating the arc at higher frequencies into a semi-circle, the diameter of which provides the Faraday, or electrochemical resistance. The curves in FIG. 5 demonstrate that the arcs at higher frequencies flatten out with the increase of Na⁺ concentration. FIG. 6 plots the electrochemical resistance (calculated from the impedance curves in FIG. 5) versus Na⁺ concentration. FIG. 6 shows that the electrochemical resistance is inversely proportional to the Na⁺ concentration. Without wishing to be bound by any particular theory, this suggests that the Faraday resistance results from Na⁺ intercalation in the antibody immobilized in the polypyrrole matrix. Again without wishing to be bound by any particular theory, since no electrochemical species was present in solution, the electrochemical reaction most likely is a polypyrrole doping process, which indicates that the ion intercalation process occurs in the antibody immobilized in the polypyrrole matrix. In the high frequency range, semicircles were observed for these responses. It is preferred to use Na⁺ or Li⁺ as the ion intercalators, however the skilled artisan will recognize that other ions (both anionic and cationic) as intercalators may be used, such as and not by way of limitation K⁺, Ca²⁺, Zn²⁺, (NH₄)⁺, Be²⁺, Mg²⁺, Ba²⁺, Al³⁺, In³⁺, Cl⁻, F⁻, Br⁻, I⁻, S²⁻, SO₄²⁻, ClO₄²⁻, Fe(CN)₆^{3-/4-}, and some organic ions. These ions can be added as alone or in mixtures in buffer solutions as supporting electrolyte.

### EXAMPLE 2

A polypyrrole/protein probe film was electrochemically deposited on microelectrode as described in Example 1. A Solartron Impedance Frequency Analyzer 1260 with Electrochemical Interface 1287 was used to measure the impedance of the microelectrode with the polypryrrole/protein probe film. A 2-electrode measurement system was used, in which the counter electrode was platinum wire. After a baseline reading, the electrode was incubated for 2 hours in 200 μL of 500 μg/mL unlabled BAP (49 kD) in PBS, at room temperature. The electrode was vigorously rinsed in PBS solution and AC impedance measurements were conducted again. The impedance results shown in FIG. 7 demonstrate that after the incubation the impedance (curve 2) changed by more than three orders of magnitude from the baseline (curve 1). The results suggest that the impedance change are due to antibody/antigen binding, as described further below.

### EXAMPLE 3

A negative control experiment was designed. A polypyrrole film deposited microelectrode without the anti-BAP probe was directly prepared in solution A by using the electrochemical synthesis procedure described in Example 1. After rinsing the polypyrrole coated electrode with PBS solution, AC impedance was measured with the same procedure described in Example 1. After a baseline reading, the electrode was incubated for 2 hours in 200 μL of 500 μg/mL unlabled BAP (49 kD) in PBS, at room temperature. The electrode was vigorously rinsed by PBS solution and AC impedance measurements were conducted again. Additionally, the experiment from Example 2 was repeated for comparison purposes. FIG. 8 plots both AC impedance results obtained from electrodes with polypyrrole/protein probe film and only the polypyrrole film. Curve 1 represents impedance of an electrode with only the polypyrrole film. Curve 2 represents impedance of the electrode of curve 1 after incubation in BAP solution. Curve 3 represents impedance of an electrode with the polypyrrole/anit-BAP probe film prior to incubation in the BAP solution. Curve 4 represent impedance of the electrode of curve 3 after incubation in the BAP solution. The results show that after the incubation the impedance of the electrode with only the polypyrrole film (curve 2) did not change significantly in comparison to the baseline (curve 1). Curves 3 and 4 further demonstrate here that anti-BAP/BAP binding causes dramatic impedance change. In addition, curve 3, the baseline for the anti-BAP/polypyrrole film, has a different impedance response from the baseline for polypyrrole, suggesting the anti-BAP were attached on the polypyrrole matrices. These results further confirm that this electrical detection method can be used to detect protein targets with polypyrrole/protein probe composite electrodes.

### EXAMPLE 4

A solution for probe synthesis was prepared by adding 1 μL 4-5 μg/mL of Virostat rabbit anti-GP120 (unconjugated), into 99 μL of solution A. Polypyrrole/protein probes films were electrochemically deposited on a microelectrode by the procedure described in Example 1. After rinsing the electrode with PBS solution, AC impedance was measured with the same procedure described in Example 1. After a baseline reading, the electrode was incubated for 1.5 hours in 200 μL of 25 μg/mL unlabled GP 120 (120 kD), an outer envelope protein of HIV-I in PBS, at room temperature. The electrode was vigorously rinsed by PBS solution and AC impedance measurements were conducted again. The results are shown in FIG. 9, demonstrating that after the incubation (curve 2) the impedance significantly changed in comparison to the baseline (curve 1).

### EXAMPLE 5

A solution for probe synthesis was prepared by adding 1 μL 100 μg/mL of anti-AZT into 99 μL of solution A. Polypyrrole/protein probes films were electrochemically deposited on a microelectrode by the procedure described in Example 1. After rinsing the electrode with PBS solution, AC impedance was measured with the same procedure described in Example 1. After a baseline reading, the electrode was incubated for 1 hour in 200 μL of 10 μg/mL AZT thymidine analog (281 D)in PBS at room temperature. The electrode was vigorously rinsed by PBS solution and AC impedance measurements were conducted again. The results are shown in FIG. 10, demonstrating that after the incubation the impedance (curve 2) significantly changed in comparison to the baseline (curve 1).

### EXAMPLE 6

A solution for probe synthesis was prepared by adding 1 μL 200 μg/mL of Intercel anti-GP 105 into 99 μL of solution A. Polypyrrole/protein probes films were electrochemically deposited on a microelectrode by the procedure described in Example 1. After rinsing the electrode with PBS solution, AC impedance was measured with the same procedure described in Example 1. After a baseline reading, the electrode was incubated for 1.0 hours in 200 μL of 10 μg/mL unlabled GP 105 (105 kD), an outer envelope protein of HIV-II, in PBS at room temperature. The electrode was vigorously rinsed by PBS solution and AC impedance measurements were conducted again. The results are shown in FIG. 11, demonstrating that after the incubation the impedance (curve 2) significantly changed in comparison to the baseline (curve 1).

It is observed (FIGS. 5-11) that different AC impedance responses were obtained from different antibody/antigen bindings. Not wishing to be bound by any particular theory, this may be caused by different structures of the antibody/antigen binding species, resulting in different cation intercalation processes. This is important in the antibody/antigen sensor applications for specificity.

### EXAMPLE 7

Polypyrrole/anti-BAP probe films were electrochemically deposited on ten microelectrodes by the procedure described in Examples 1 and 2. Solutions of 0.01, 0.1, 1, 10 and 100 ng/mL BAP in 1× PBS were prepared. After rinsing the electrodes with PBS solution, AC impedance of the ten electrodes was measured with the same procedure described in Example 1 to provide a baseline. Two electrodes were incubated in each of the 5 BAP solutions for 2 hours. The electrodes were rinsed vigorously in a PBS solution and impedance measurements were taken of each electrode, as described in the Examples above. The change in impedance resistance for each electrode before and after incubation was calculated by calculating the diameter of the impedance curve at the higher frequencies, as described above. Referring to FIG. 12, the average of the change in impedance resistance for each of the five BAP solutions is plotted versus the concentration of each of the solutions.

These results demonstrate that the present method and apparatus using reporterless electrochemical detection can detect the presence of target analyte (BAP) down to at least 10 pg/ml (approx. 100 fM), with a dynamic range of over three-orders of magnitude to reach a plateau response.

### EXAMPLE 8

Polypyrrole/anti-BAP probe films were electrochemically deposited on four microelectrodes by the procedure described in Examples 1 and 2. A solution of 10 ng/mL BAP and a solution of 10 ng/mL gp120 in 1× PBS were prepared. After rinsing the electrodes with PBS solution, AC impedance of the four electrodes was measured at frequencies ranging from 0.1 Hz to 1 MHz with the same procedure described in Example 1 to provide a baseline, from which an average impedance resistance at each frequency was calculated, as described above. Two of the electrodes were incubated in the BAP solution and the other two were incubated in the gp120 solution for two hours. The electrodes were rinsed vigorously in a PBS solution and impedance measurements were taken of each electrode at frequencies ranging from 0.1 Hz to 1 MHz, as described in the Examples above, from which an average impedance resistance at each frequency was calculated. Referring to FIG. 13, the average of the change in impedance resistance for each pair of electrodes is plotted versus the inverse of the square-root of frequency at which the measurement was taken. These results show that the crossreaction rate of gp120 antigen to the polypyrrole/anti-BAP probe is 0.08, demonstrating excellent specificity of this labelless detection technology.

What is claimed is:

1. A method for the electrical detection of the presence of a target molecule based upon a molecular interaction between a probe molecule and said target molecule, the method comprising:
(a) measuring an electrical property of a microelectrode, wherein said microelectrode is in contact with a conductive polymer matrix having a molecular probe immobilized thereto;
(b) exposing said microelectrode to a sample mixture suspected of containing said target molecules;
(c) measuring said electrical property of said microelectrode; and
(d) comparing the measurement of step (a) to the measurement in step (c), thereby determining whether a molecular interaction between said probe and said target molecule occurred, and thereby confirming the presence of said target molecule in said sample mixture.

2. The method according to claim 1, wherein said probe molecule is a protein or peptide.

3. The method according to claim 2, wherein said target molecule is a small molecule.

4. The method according to claim 1, wherein said target molecule is a protein or peptide.

5. The method according to claim 4, wherein said probe molecule is a protein or peptide.

6. The method according to claim 1, wherein said probe molecule is immobilized within said conductive polymer matrix.

7. The method according to claim 6, wherein said probe molecule is covalently immobilized within said conductive polymer matrix.

8. The method according to claim 1, wherein said probe molecule is covalently immobilized on said conductive polymer matrix.

9. The method according to claim 1, wherein said microelectrode is an array of microelectrodes.

10. The method according to claim 1, wherein the interaction between said protein or peptide target molecule and said probe is enhanced in the presence of an ionic intercalator in said sample mixture.

11. The method according to claim 10, wherein said ionic intercalator is selected from the group consisting of K⁺, Na⁺, Li⁺Ca²⁺, Zn²⁺, (NH₄)⁺, Be²⁺, Mg²⁺, Ba²⁺, Al³⁺, In³⁺, Cl⁻, F⁻, Br⁻, I⁻, S²⁻, SO₄²⁻, ClO₄²⁻, and Fe(CN)₆^{3-/4-}.

12. The method according to claim 10, wherein said ionic intercalator is a cation.

13. The method according to claim 12, wherein said cation is selected from the group consisting of K⁺, Na⁺, Li⁺Ca²⁺, Zn²⁺, (NH₄)⁺, Be²⁺, Mg²⁺, Ba²⁺, Al³⁺, and In³⁺.

14. The method according to claim 12, wherein said cation is selected from the group consisting of K⁺, Na⁺, and Li⁺.

15. The method according to claim 1, wherein said conductive polymer matrix is selected from the group consisting of polypyrrole, polythiphene, polyaniline, polyfuran, polypyridine, polycarbazole, polyphenylene, poly(phenylenevinylene), polyfluorene, polyindole, derivatives thereof, co-polymers thereof, and combinations thereof.

16. The method according to claim 15, wherein said conductive polymer is selected from the group consisting of polypyrrole, polythiophene and polyaniline.

17. A apparatus for the electrical detection of the presence of a protein or peptide target molecule based upon a molecular interaction between a probe molecule and said protein or peptide target molecule, the apparatus comprising:
(a) a microelectrode; and
(b) a conductive polymer matrix with a probe molecule immobilized thereto, wherein said polymer matrix is in contact with said microelectrode, and wherein a molecular interaction between a protein or peptide target molecule and said probe molecule in an electrolytic solution results in a change of an electrical property of said microelectrode.

18. The apparatus according to claim 17, wherein said probe molecule is immobilized within said conductive polymer matrix.

19. The apparatus according to claim 18, wherein said probe molecule is covalently immobilized within said conductive polymer matrix.

20. The apparatus according to claim 17, wherein said probe molecule is covalently immobilized on said conductive polymer matrix.

21. The apparatus according to claim 17, wherein said electrical property is impedance.

22. The apparatus according to claim 17, wherein the interaction between said protein or peptide target molecule and said probe is enhanced in the presence of an ionic intercalator in said electrolytic solution.

23. The apparatus according to claim 22, wherein said ionic intercalator is selected from the group consisting of K⁺, Na⁺, Li⁺ Ca²⁺, Zn²⁺, (NH₄)⁺, Be²⁺, Mg²⁺, Ba²⁺, Al³⁺, In³⁺, Cl⁻, F⁻, Br⁻, I⁻, S²⁻, SO₄²⁻, ClO₄²⁻, and Fe(CN)₆^{3-/4-}.

24. The apparatus according to claim 23, wherein said ionic intercalator is a cation.

25. The apparatus according to claim 24, wherein said cation is selected from the group consisting of K⁺, Na⁺, Li⁺Ca²⁺, Zn²⁺, (NH₄)⁺, Be²⁺, Mg²⁺, Ba²⁺, Al³⁺, and ln³⁺.

26. The apparatus according to claim 25, wherein said cation is selected from the group consisting of K⁺, Na⁺, and Li⁺.

27. The apparatus according to claim 17, wherein said target molecule is a small molecule.

28. The apparatus according to claim 27, wherein said target molecule is AZT.

29. The apparatus according to claim 17, wherein said probe molecule is a protein molecule selected from the group consisting of an antibody and an antigen.

30. The apparatus according to claim 17, wherein said probe molecule is a protein or peptide.

31. The apparatus according to claim 30, wherein said target molecule is a small molecule.

32. The apparatus according to claim 17, wherein said target molecule is a protein or peptide.

33. The apparatus according to claim 32, wherein said probe molecule is a protein or
